(19) United States
(12) Patent Application Publication
(10) Pub. No.: US 2012/0028260 A1
(43) Pub. Date: Feb. 2, 2012

(54) RAD9 AS A DIAGNOSTIC, PROGNOSTIC AND THERAPEUTIC TOOL FOR PROSTATE CANCER

(75) Inventors: Howard B. Lieberman, Tenafly, NJ (US); Aiping Zhu, Fort Lee, NY (US)

(73) Assignee: The Trustees of Columbia University In The City of New York

(21) Appl. No.: 13/220,422

(22) Filed: Aug. 29, 2011

Related U.S. Application Data
(63) Continuation of application No. 12/451,881, filed on Feb. 5, 2010.
(60) Provisional application No. 60/933,336, filed on Jun. 6, 2007.
(60) Provisional application No. 61/067,717, filed on Feb. 29, 2008.

(51) Int. Cl. C12Q 1/68; G01N 21/64; G01N 33/577; G01N 33/574

(52) U.S. Cl. 435/6.12; 436/501; 435/7.1

(57) ABSTRACT

This disclosure provides a method of treating a human subject having a cancer which comprises administering to the subject a nucleic acid so as to inhibit expression of human RAD9 protein in a cell of the cancer so as to thereby treat the human subject. This disclosure also provides methods of assessing gains in prostate cancer therapy and detecting prostate cancer.

This application is a continuation of U.S. Ser. No. 12/451,681, §371 national stage of PCT International Application No. PCT/US2008/007142, filed Jun. 6, 2008, which claims priority of U.S. Provisional Application No. 61/067,717, filed Feb. 29, 2008 and U.S. Provisional Application No. 60/933,336, filed Jun. 6, 2007, the contents of each of which are hereby incorporated by reference in their entireties into this application.

Throughout this application, various publications are referenced in parentheses by name or number. Full citations for the references cited by number may be found at the end of each experimental section. The disclosures of all of these publications in their entireties are hereby incorporated by reference into this application to more fully describe the state of the art to which this disclosure pertains.

This invention was made with government support under grant numbers CM079107, CA130536, and GM52493 awarded by the National Institutes of Health, U.S. Department of Health and Human Service. The government has certain rights in the invention.

### BACKGROUND OF THE DISCLOSURE

Prostate cancer is a very common type of cancer in American men. The American Cancer Society estimates that 234,460 new cases will arise in 2006 in the United States, and 27,350 men will die because of it. African-American men get and die from this cancer more frequently than white or Asian men, but the reasons why are not clear. Therefore, the development of a cure is of great concern and urgency. There are a number of treatment options available for prostate cancer, including surgery, radiation, chemotherapy, hormone therapy, and several alternative medicine approaches. Another option is simply to “wait and watch”. However, the final selection is based on multiple factors, including age, general health, grade of the existing cancer, whether it has remained localized in prostate tissue, and potential side effects.

Human RADS (HRAD9) is an evolutionarily conserved human gene first identified by us as a genetic element important for promoting resistance to DNA damage and regulating cell cycle checkpoints (see U.S. Pat. No. 5,882,862, issued Mar. 16, 1999, hereby incorporated by reference). Subsequent analyses of this gene indicated that it had a much broader range of activities. The encoded protein can induce apoptosis, as well as regulate genomic stability. In addition, it has 3′ to 5′ exonuclease activity, the ability to bind p53 consensus DNA binding sequences and upregulate transcription of p21 as well as other downstream genes, the ability to stimulate the carbamoyl phosphate synthetase activity of CAD protein, required for de novo synthesis of pyrimidine nucleotides and cell growth, and the ability to associate with and stimulate the activity of several DNA repair proteins.

### SUMMARY OF THE DISCLOSURE

A method of treating a subject having a cancer which comprises administering to the subject a nucleic acid which inhibits expression of a human RAD9 gene so as to thereby treat the human subject.

A method of treating a subject having a cancer which comprises administering to the subject an agent which decreases methylation of a human RAD9-encoding nucleic acid in a cell of the cancer so as to thereby treat the subject.

A composition comprising (i) a short interfering nucleic acid directed to a nucleic acid encoding human RAD9 and (ii) a carrier.

A composition comprising (i) a vector comprising a nucleic acid encoding a short interfering nucleic acid directed to a nucleic acid encoding human RAD9 and (ii) a carrier.

A method of determining the likelihood that a human subject is suffering from a cancer of a prostate comprising:
a) obtaining a sample from the prostate of the human subject;
b) quantitating the amount of human Rad9 in the sample; and
c) comparing the amount of human Rad9 quantitated in step b) with a reference amount,
wherein an amount of human Rad9 quantitated in step b) greater than the reference amount indicates that the human subject is likely suffering from a cancer of the prostate.

A method of determining if a cancer of a prostate of a human subject is metastatic comprising:
obtaining a sample from the cancer of the prostate of the human subject;
quantitating the amount of human Rad9 in the sample; and
comparing the amount of human Rad9 quantitated in step b) with a reference amount,
wherein an amount of human Rad9 quantitated in step b) greater than the reference amount indicates that the cancer of the prostate is metastatic.

A method of determining the likelihood that a human subject is suffering from a cancer of a prostate comprising:
a) obtaining a sample from the prostate of the human subject;
b) quantitating the amount of human Rad9-encoding messenger RNA in the sample; and
c) comparing the amount of human Rad9-encoding messenger RNA quantitated in step b) with a reference amount,
wherein an amount of human Rad9-encoding messenger RNA quantitated in step b) greater than the reference amount indicates that the human subject is likely suffering from a cancer of the prostate.

A method of determining if a cancer of a prostate of a human subject is metastatic comprising:
a) obtaining a sample from the cancer of the prostate of the human subject;
b) quantitating the amount of human Rad9-encoding messenger RNA in the sample; and
c) comparing the amount of human Rad9-encoding messenger RNA quantitated in step b) with a reference amount,
wherein an amount of human Rad9-encoding messenger RNA quantitated in type b) greater than the reference amount indicates that the cancer of the prostate is metastatic.

A method of determining whether a therapy is efficacious in treating a cancer of the prostate of a human subject comprising:
a) obtaining a sample from the prostate of the human subject;
b) quantitating the amount of human Rad9 in the sample;
c) treating the human subject with the therapy;
d) obtaining a sample from the prostate of the human subject who has been treated with the therapy;
e) quantitating the amount of human Rad9 in the sample; and
f) comparing the amount of human Rad9 quantitated in step b) with the amount of human Rad9 quantitated in step e),
wherein an amount of human Rad9 quantitated in step e) less than the amount of human Rad9 quantitated in step b) indicates that the therapy is efficacious in treating the cancer of the prostate.

A method of determining whether a therapy is efficacious in treating a cancer of the prostate of a human subject comprising:
a) obtaining a sample from the prostate of the human subject;
b) quantitating the amount of human Rad9-encoding messenger RNA in the sample;
c) treating the human subject with the therapy;
d) obtaining a sample from the prostate of the human subject who has been treated with the therapy;
e) quantitating the amount of human Rad9-encoding messenger RNA in the sample; and
f) comparing the amount of human Rad9-encoding messenger RNA quantitated in step b) with the amount of human Rad9-encoding messenger RNA quantitated in step e),
wherein an amount of human Rad9-encoding messenger RNA quantitated in step e) less than the amount of human Rad9-encoding messenger RNA quantitated in step b) indicates that the therapy is efficacious in treating the cancer of the prostate.

### BRIEF DESCRIPTION OF THE FIGURES

FIGS. 1A and 1B: (A) Western blot of Rad9 protein levels elevated in human prostate cancer cells. PrEC is a normal human prostate cell population, whereas PC3, LNCaP, DU145 and CWR22 are prostate cancer cell lines. Actin is indicated and serves as a loading control. (B) graph of Rad-9/beta-actin band intensity ratios for the samples in 1A.

FIG. 2: Immunohistochemical staining for human Rad9 protein in normal and prostate cancer cells. PrEC is a noncancerous prostate cell population and the rest are cancer cells. Note the heavily stained (black) nuclei indicative of Rad9 in the cancer cell lines, relative to the more lightly stained PrEC control (gray).

FIG. 3: Rad9 gene in PC3 cells might be amplified. Top: Southern blot indicating intensities of Rad9 and Beta-actin DNA bands in cells; Bottom: Ratios of Rad9 to Beta-actin bands quantified and presented relative to the ratio in PrEC. Data represent the average of three independent trials, ±S.D.

FIG. 4: Methylation of Rad9 CpG islands in normal and prostate cancer cells. Black dots: cytosine methylation within the designated CpG island. E1, Exon 1; I1, Intron 1; E2, Exon 2; I2, Intron 2. The start of translation, denoted as +1, is the beginning of the first ATG in Exon 1. DU145 Rad9 is aberrantly hypermethylated.

FIG. 5: Methylation of Rad9 CpG islands in normal and prostate cancer cells treated with 5′-aza-2′-deoxycytidine. See FIG. 4 legend for experimental details.

FIG. 6: 5′-aza-2′-deoxycytidine treatment reduces Rad9 protein levels in DU145 but not in PrEC, PC3, LNCaP or CWR22 cells. These results indicate that aberrant methylation is responsible for the abnormally high levels of Rad9 protein in DU145 cells. Beta-actin control.

FIGS. 7A-7C: Western blots indicating reduction of HRAD9 protein in prostate cancer cells. 7A: DU145 cells untransfected, with insertless vector, and 2 stable clones with HRAD9 siRNAs; 73: PC3 cells, same as 7A but showing only 1 independent siRNA clone; 7C: CWR22 cells, similar to 7A. HRAD9 siRNA was most effective in reducing levels of the protein in DU145 and PC3 cells.

FIGS. 8A-8C: Reduction of HRAD9 levels in prostate cancer cells reduce tumorigenicity. Cells were injected into multiple sites in the backs of mice subcutis. Tumor formation was monitored up to 35 days, except for CWR22 where tumors grew so aggressively that the experiment was terminated on day 25. 8A: DU145 cells with insertless vector or HRAD9 siRNA; 8B: PC3 cells as per 8A; 8C: Injection of CWR22 cells as per 8A. There was a direct dose-dependent relationship between HRAD9 levels and tumor growth; the less Rad9 the fewer tumors. Each bar is a single site injection. Sites injected with DU145 cells+Rad9 siRNA were monitored for five months, beyond the data shown, and still no tumors formed. Arrows above bars indicate empty vector control or siRNA transformants.

FIG. 9: Human RAD9 protein sequence (SEQ ID NO:1). NCBI Protein Accession No. NP_{—}004575. SNPs give rise to variants with a glutamine at residue 71 (SEQ ID NO:2) or an alanine at residue 100 (SEQ ID NO:3).

FIG. 10: A nucleotide sequence encoding Human RAD9 protein sequence (SEQ ID NO:4). NCBI Nucleotide Accession No. NM_{—}004504, SNPs include NCBI refSNP ID: rs17881103, refSNP ID: rs3832777 and refSNP ID: rs2066495.

FIG. 11: Immunohistochemical staining for HRAD9 protein in thin sections of prostate tissue. Panels: (A) Adenocarcinoma, Stage III, weak staining (+) for HRAD9 protein; (B) Adenocarcinoma, Stage III, strong staining (++) for HRAD9; (C) Adenocarcinoma, Stage IV, very intense stain (+++); (D) Normal, noncancerous prostate tissue, no detectable HRAD9 stain. Brown stain, HRAD9 protein; Blue areas, negative.

### DETAILED DESCRIPTION OF THE INVENTION

A method of treating a subject having a cancer which comprises administering to the subject a nucleic acid which inhibits expression of a human RAD9 gene so as to thereby treat the human subject.

In an embodiment the cancer is a cancer of the prostate.

in an embodiment the nucleic acid is, or upon transcription becomes, a short interfering ribonucleic acid. In an embodiment the short interfering ribonucleic acid comprises two ribonucleic acid strands, one of which comprises about 15 to about 28 ribonucleotides the sequence of which is complementary to a sequence of consecutive nucleotides present within the human RAD9 gene, and the other of which comprises about 15 to about 28 ribonucleotides, the sequence of which is identical to such sequence of consecutive nucleotides within the human RAD9 gene. In an embodiment the sequence of consecutive nucleotides present within the human RAD9 gene comprises AGGCCCGCCAUCUUCACCA (SEQ ID NO:5). In an embodiment each strand of the short interfering ribonucleic is 21 nucleotides in length, and wherein the sequence of 19 consecutive nucleotides of one of the strands is complementary to a sequence of 19 consecutive nucleotides present within the human RAD9 gene, and wherein the sequence of the other strand is 21 ribonucleotides in length, the sequence of 19 consecutive nucleotides of which is identical to such sequence of consecutive nucleotides within the human RAD9 gene. In an embodiment the two strands of the short interfering ribonucleic acid are base paired for 19 consecutive nucleotides and have a 2-nucleotide overhang at their respective 3′ ends. In an embodiment one or more of the ribonucleotides is modified in a sugar or base present therein. In an embodiment at least one of the strands comprises an inter-ribonucleotide phosphorothioate bond.

In an embodiment the nucleic acid is a hairpin ribonucleic acid.

In an embodiment the nucleic acid is administered to the subject by injection into the prostate of the subject. In an embodiment the injection into the prostate of the subject is effected via a catheter into the prostate of the subject. In an embodiment administering the nucleic acid to the subject is effected by administering the subject a vector comprising the nucleic acid. In an embodiment the nucleic acid is transcribed in a cell of the subject into a short interfering ribonucleic acid. In an embodiment the vector comprises a RNA III polymerase promoter. In an embodiment the RNA III polymerase promoter is a U6 promoter or a H1 promoter. In an embodiment the vector comprises a RNA III polymerase termination site. In an embodiment the termination site is a T5 sequence. In an embodiment the nucleic acid is transcribed in a cell of the subject into a short hairpin ribonucleic acid.

In an embodiment the method further comprises irradiating the cancer with radiation from a radiation source. In an embodiment the radiation source is a radioisotope or external beam radiation. In an embodiment the external beam radiation is from a linear accelerator. In an embodiment the method further comprises the radiation source is a radioisotope which is Iodine 125 or Palladium 103.

In an embodiment the method further comprises reducing the amount of an androgen present in the prostate. In an embodiment the androgen is testosterone or 5-alpha-dihydrotestosterone. In an embodiment the amount of androgen is reduced by administering an androgen-suppressing drug to the subject. In an embodiment the androgen-suppressing drug is a lutenizing hormone-releasing hormone receptor agonist. In an embodiment the lutenizing hormone-releasing hormone receptor agonist is leuprolide acetate or goserelin acetate.

In en embodiment the method further comprises reducing methylation of a human RAD9-encoding nucleic acid in a cell of the cancer.

A method of treating a subject having a cancer which comprises administering to the subject an agent which inhibits expression of a human RAD9 gene so as to thereby treat the human subject.

In an embodiment the cancer is a cancer of the prostate.

In an embodiment the agent is a small molecule. In an embodiment the agent is an antibody directed against human RAD9. In an embodiment the antibody is a monoclonal antibody. In an embodiment the antibody is a humanized antibody. In an embodiment the agent is a fusion protein directed against a RAD9 receptor. In an embodiment the agent is identified by an assay which comprises exposing a prostate cell which expresses RAD9 (for example a transformed prostate cell, prostate cancer cell line) to the agent and then determining the radiosensitivity and RAD9 expression level (e.g. via mRNA copy number or by protein expression level) of the cell in the presence of the agent, or after exposure to the agent. The radiosensitivity and RAD9 expression are compared to the radiosensitivity and RAD9 expression of the same cell-type which is not (or has not been) exposed to the agent. An increase in radiosensitivity and decrease in RAD9 expression in the presence of (or after exposure to) the agent indicates that the agent is inhibits expression of a human RAD9 gene so as to render a cell expressing the RAD9 susceptible to radiotherapy.

An “antibody” shall include, without limitation, an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. “Antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Methods for humanizing antibodies are well known to those skilled in the art. “Antibody” also includes, without limitation, a fragment or portion of any of the afore-mentioned immunoglobulin molecules and includes a monovalent and a divalent fragment or portion. Antibody fragments include, for example, Fc fragments and antigen-binding fragments (Fab).

“Monoclonal antibodies,” also designated a mAbs, are antibody molecules whose primary sequences are essentially identical and which exhibit the same antigenic specificity. Monoclonal antibodies may be produced by hybridoma, recombinant, transgenic or other techniques known to those skilled in the art.

A “humanized” antibody refers to an antibody wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules, whereas some, most or all amino acids within one or more CDR regions are unchanged. Smell additions, deletions, insertions, substitutions or modifications of amino acids are permissible as long as they do not abrogate the ability of the antibody to bind a given antigen. Suitable human immunoglobulin molecules include IgG1, IgG2, IgG3, IgG4, IgA, IgE and IgM molecules. A “humanized” antibody retains an antigenic specificity similar to that of the original antibody.

One skilled in the art would know how to make the humanized antibodies of the subject invention. Various publications, several of which are hereby incorporated by reference into this application, also describe how to make humanized antibodies. For example, the methods described in U.S. Pat. No. 4,816,567 (71) comprise the production of chimeric antibodies having a variable region of one antibody and a constant region of another antibody.

U.S. Pat. No. 5,225,539 (72) describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Antibodies directed against the sequences of RAD9 disclosed herein are encompassed within the scope of the invention.

A method of treating a subject having a cancer which comprises administering to the subject an agent which decreases methylation of a human RAD9-encoding nucleic acid in a cell of the cancer so as to thereby treat the subject.

In an embodiment the agent is 5′-aza-2′ deoxycytidine. In an embodiment the agent is administered by injection, catheterization, heat shock or electroporation. In an embodiment the cancer is a cancer of the prostate. In an embodiment the agent is administered by direct injection or catheterization into a prostate gland of the subject.

A composition comprising (i) a short interfering nucleic acid directed to a nucleic acid encoding human RAD9 and (ii) a carrier.

A composition comprising (i) a vector comprising a nucleic acid encoding a short interfering nucleic acid directed to a nucleic acid encoding human RAD9 and (ii) a carrier.

in an embodiment the composition further comprises an anti-cancer agent. In an embodiment the anti-cancer agent is a radioactive source. In an embodiment the radioactive source is a radioisotope. In an embodiment the anti-cancer agent is an androgen-suppressing drug.

In an embodiment the composition further comprises a de-methylating agent.

In an embodiment the carrier is a pharmaceutically acceptable carrier.

A method of determining the likelihood that a human subject is suffering from a cancer of a prostate comprising:
a) obtaining a sample from the prostate of the human subject;
b) quantitating the amount of human Rad9 in the sample; and
c) comparing the amount of human Rad9 quantitated in step b) with a reference amount,
wherein an amount of human Rad9 quantitated in step b) greater than the reference amount indicates that the human subject is likely suffering from a cancer of the prostate.

A method of determining if a cancer of a prostate of a human subject is metastatic comprising:
obtaining a sample from the cancer of the prostate of the human subject;
quantitating the amount of human Rad9 in the sample; and
comparing the amount of human Rad9 quantitated in step b) with a reference amount,
wherein an amount of human Rad9 quantitated in step b) greater than the reference amount indicates that the cancer of the prostate is metastatic.

In embodiments, the amount of human Rad9 is quantitated by immunochemistry, immunofluorescence or immunoradiology.

A method of determining the likelihood that a human subject is suffering from a cancer of a prostate comprising:
a) obtaining a sample from the prostate of the human subject;
b) quantitating the amount of human Rad9-encoding messenger RNA in the sample; and
c) comparing the amount of human Rad9-encoding messenger RNA quantitated in step b) with a reference amount,
wherein an amount of human Rad9-encoding messenger RNA quantitated in step b) greater than the reference amount indicates that the human subject is likely suffering from a cancer of the prostate.

A method of determining if a cancer of a prostate of a human subject is metastatic comprising:
a) obtaining a sample from the cancer of the prostate of the human subject;
b) quantitating the amount of human Rad9-encoding messenger RNA in the sample; and
c) comparing the amount of human Rad9-encoding messenger RNA quantitated in step b) with a reference amount,
wherein an amount of human Rad9-encoding messenger RNA quantitated in type b) greater than the reference amount indicates that the cancer of the prostate is metastatic.

A method of determining whether a therapy is efficacious in treating a cancer of the prostate of a human subject comprising:
a) obtaining a sample from the prostate of the human subject;
b) quantitating the amount of human Rad9 in the sample;
c) treating the human subject with the therapy;
d) obtaining a sample from the prostate of the human subject who has been treated with the therapy;
e) quantitating the amount of human Rad9 in the sample; and
f) comparing the amount of human Rad9 quantitated in step b) with the amount of human Rad9 quantitated in step e),
wherein an amount of human Rad9 quantitated in step e) less than the amount of human Rad9 quantitated in step b) indicates that the therapy is efficacious in treating the cancer of the prostate.

A method of determining whether a therapy is efficacious in treating a cancer of the prostate of a human subject comprising:
a) obtaining a sample from the prostate of the human subject;
b) quantitating the amount of human Rad9-encoding messenger RNA in the sample;
c) treating the human subject with the therapy;
d) obtaining a sample from the prostate of the human subject who has been treated with the therapy;
e) quantitating the amount of human Rad9-encoding messenger RNA in the sample; and
f) comparing the amount of human Rad9-encoding messenger RNA quantitated in step b) with the amount of human Rad9-encoding messenger RNA quantitated in step e),
wherein an amount of human Rad9-encoding messenger RNA quantitated in step e) less than the amount of human Rad9-encoding messenger RNA quantitated in step b) indicates that the therapy is efficacious in treating the cancer of the prostate.

In embodiments the human Rad9 comprises consecutive amino acid residues having the sequence set forth in SEQ ID NO:1, SEQ ID NO:2 or SEQ ID NO:3. In embodiments the amount of human Rad9-encoding messenger RNA is quantitated by reverse transcriptase polymerase chain reaction. In embodiments the human Rad9-encoding messenger RNA comprises consecutive ribonucleotides corresponding to the DNA sequence set forth in SEQ ID NO:4.

As used herein, “reference amount” means a normalized value obtained from a normal sample, and in the case of RAD9 protein means the amount of RAD9 protein measured from a non-cancerous or other standardized sample (normalized for mass/size) as measured by a parallel assay with the same steps and conditions to which the tested or cancerous sample is being subjected.

As used herein, a “pharmaceutically acceptable carrier” is one that is suitable for use with humans and/or animals without undue adverse side effects (such as toxicity, irritation, and allergic response) commensurate with a reasonable benefit/risk ratio.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this disclosure. For example, an amount effective to delay the growth of or to cause a cancer to shrink or not metastasize. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“siRNA” shall mean small interfering ribonucleic acid, i.e. a short (e.g. 21-23 nt) RNA duplex which can elicit an RNA interference (RNAi) response in a mammalian cell. siRNAs may be blunt ended or have mono, di or trinucleotide 3′ overhangs.

“shRNA” shall mean short hairpin interfering ribonucleic acid containing a double stranded base-paired segment, each strand of which is contiguous at one of its ends with a loop (or non-base-paired) segment and which can be processed in a cell into a siRNA. By way of example, the base-paired segment can be 19 base-pairs in length.

“Amino acid residue” shall mean one of the individual monomer units of a peptide chain, which result from at least two amino acids combining to form a peptide bond.

“Amino acid” shall mean an organic acid that contains both a basic amino group, an acidic carboxyl group and an R group.

“Complementary” with regard to a nucleic acid sequence shall mean fully matching a sequence by base-pairing.

This disclosure relates to compounds, compositions, and methods useful for modulating RAD9 gene expression using short interfering nucleic acid (siNA) molecules. This disclosure also relates to compounds, compositions, and methods useful for modulating the expression and activity of other genes involved in pathways of RAD9 gene expression and/or activity by RNA interference (RNAi) using small nucleic acid molecules. In particular, the instant disclosure features small nucleic acid molecules, such as short interfering nucleic acid (siNA), short interfering RNA (siRNA), double-stranded RNA (dsRNA), micro-RNA (miRNA), and short hairpin ANA (shRNA) molecules and methods used to modulate the expression of RAD9 genes, including human RAD9.

A siNA of the disclosure can be unmodified or chemically-modified. A siNA of the instant disclosure can be chemically synthesized, expressed/transcribed from a vector or enzymatically synthesized. The instant disclosure also features various chemically-modified synthetic short interfering nucleic acid (siNA) molecules capable of modulating target gene expression or activity in cells by RNA interference (RNAi). The use of chemically-modified siNA improves various properties of native siNA molecules through increased resistance to nuclease degradation in vivo and/or through improved cellular uptake. siNA having multiple chemical modifications retains its RNAi activity.

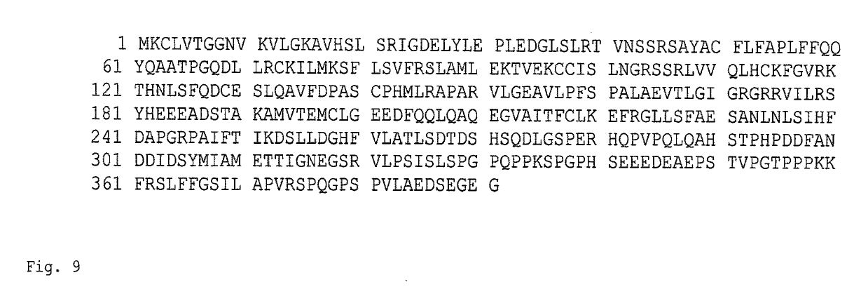

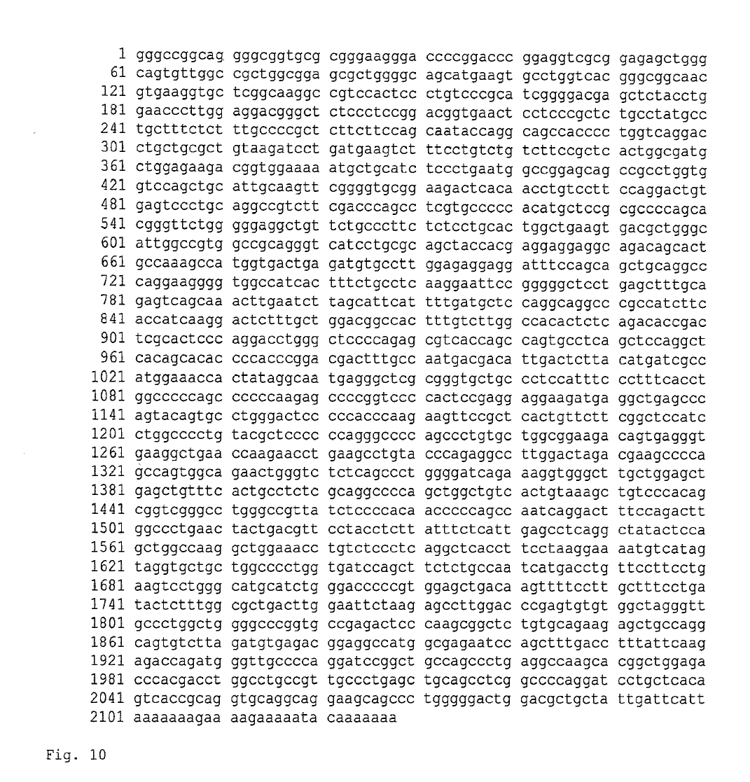

In one embodiment, the disclosure features one or more siNA molecules and methods that independently or in combination modulate the expression of a RAD9 gene encoding a RAD9 protein. In one embodiment the RAD9 encoded by the RAD9 gene is human RAD9. In one embodiment the RAD9 comprises consecutive amino acids having the sequence set forth in FIG. 9 (SEQ ID NO:1). In different embodiments the encoded RAD9 is a variant of SEQ ID NO:1, such as the sequences set forth in SEQ ID NO:2 or SEQ ID NO:3, see FIG. 9. The description below of the various aspects and embodiments of the disclosure is provided with reference to RAD9 target genes referred to herein as gene targets. However, the various aspects and embodiments are also directed to other genes, such as RAD9 gene homologs, transcript variants, and polymorphisms (e.g., single nucleotide polymorphism, for example SNPs include NCBI refSNP ID: rs17881103, refSNP ID: rs3832777 and refSNP ID: rs2066495). In one embodiment the siNA molecules are directed to the RNA corresponding to SEQ ID NO:4 (see FIG. 10) or the reverse complemented strand thereof. In one embodiment the siNA molecules are directed to SEQ ID NO:5. As such, the various aspects and embodiments are also directed to other genes that are involved in disease, trait, condition, or disorder related pathways of signal transduction or gene expression that are involved, for example, in the maintenance or development of diseases, traits, conditions, or disorders described herein.

In one embodiment, the disclosure features a double-stranded short interfering nucleic acid (siNA) molecule that down-regulates expression of a target gene or that directs cleavage of a target RNA, wherein said siNA molecule comprises about 15 to about 28 base pairs. In one embodiment, the disclosure features a double stranded short interfering nucleic acid (siNA) molecule that directs cleavage of a target RNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the target RNA for the siNA molecule to direct cleavage of the target RNA via RNA interference, and the second strand of said siNA molecule comprises nucleotide sequence that is complementary to the first strand.

In one embodiment, the disclosure features a double stranded short interfering nucleic acid (siNA) molecule that directs cleavage of a target RNA via RNA interference (RNAi), wherein the double stranded siNA molecule comprises a first and a second strand, each strand of the siNA molecule is about 18 to about 23 nucleotides in length, the first strand of the siNA molecule comprises nucleotide sequence having sufficient complementarity to the target RNA for the siNA molecule to direct cleavage of the target RNA via RNA interference, and the second strand of said siNA molecule comprises nucleotide sequence that is complementary to the first strand.

In one embodiment, the disclosure features a chemically synthesized double stranded short interfering nucleic acid (siNA) molecule that directs cleavage of a target RNA via RNA interference (RNAi), wherein each strand of the siNA molecule is about 18 to about 28 nucleotides in length; and one strand of the siNA molecule comprises nucleotide sequence having sufficient complementarity to the target RNA for the siNA molecule to direct cleavage of the target RNA via RNA interference.

In one embodiment, the disclosure features a chemically synthesized double stranded short interfering nucleic acid (siNA) molecule that directs cleavage of a target RNA via RNA interference (RNAi), wherein each strand of the siNA molecule is about 18 to about 23 nucleotides in length; and one strand of the siNA molecule comprises nucleotide sequence having sufficient complementarity to the target RNA for the siNA molecule to direct cleavage of the target RNA via RNA interference.

In one embodiment, the disclosure features a siNA molecule that down-regulates expression of a target gene or that directs cleavage of a target RNA, for example, wherein the gene comprises protein encoding sequence. In one embodiment, the disclosure features a siNA molecule that down-regulates expression of a target gene or that directs cleavage of a target RNA, for example, wherein the gene comprises non-coding sequence or encodes sequence of regulatory elements involved in gene expression (e.g. non-coding RNA).

In one embodiment, the disclosure features a siNA molecule having RNAi activity against target RAD9 RNA e.g., ceding or non-coding RNA), wherein the siNA molecule comprises a sequence complementary to any RNA sequence encoding a RAD9 or portion thereof. In another embodiment, the disclosure features a siNA molecule having RNAi activity against target RNA, wherein the siNA molecule comprises a sequence complementary to an RNA having variant encoding sequence

Chemical modifications can be applied to any siNA construct of the disclosure. In another embodiment, a siNA molecule of the disclosure includes a nucleotide sequence that can interact with nucleotide sequence of a target gene and thereby mediate silencing of gene expression, for example, wherein the siNA mediates regulation of gene expression by cellular processes that modulate the chromatin structure or methylation patterns of the gene and prevent transcription of the gene.

In one embodiment of the disclosure a siNA molecule comprises an antisense strand comprising a nucleotide sequence that is complementary to a target polynucleotide sequence or a portion thereof. The siNA further comprises a sense strand, wherein said sense strand comprises a nucleotide sequence of a target polynucleotide sequence or a portion thereof, (e.g., about 15 to about 25 or more, or about 15, 16, 17, 15, 19, 20, 21, 22, 23, 24, or or more contiguous nucleotides in a target polynucleotide sequence). In one embodiment, the target polynucleotide sequence is a target DNA. In one embodiment, the target polynucleotide sequence is a target RNA.

In one embodiment, the disclosure features a siNA molecule comprising a first sequence, for example, the antisense sequence of the siNA construct, complementary to a sequence or portion of sequence comprising sequence encoding RAD9, and a second sequence, for example a sense sequence, that is complementary to the antisense sequence.

In one embodiment of the disclosure a siNA molecule comprises an antisense strand having about 15 to about 30 (e.g. 15, 16, 17, 18, 19, 20, 21, 22, 23, 24, 25, 26, 27, 28, 29, or 30) nucleotides, wherein the antisense strand is complementary to a target RNA sequence or a portion thereof, and wherein said siNA further comprises a sense strand having about 15 to about 30 (e.g. 15, 16, 17, 18, 19, 20, 21, 22, 23, 24, 25, 26, 27, 28, 29, or 30) nucleotides, and wherein said sense strand and said antisense strand are distinct nucleotide sequences where at least about 15 nucleotides in each strand are complementary to the other strand.

In another embodiment of the disclosure a siNA molecule of the disclosure comprises an antisense region having about 15 to about 30 (e.g. 15, 16, 17, 18, 19, 20, 21, 22, 23, 24, 25, 26, 27, 28, 29, or 30) nucleotides, wherein the antisense region is complementary to a target DNA sequence, and wherein said siNA further comprises a sense region having about 15 to about 30 (e.g. 15, 16, 17, 18, 19, 20, 21, 22, 23, 24, 25, 26, 27, 28, 29, or 30) nucleotides, wherein said sense region and said antisense region are comprised in a linear molecule where the sense region comprises at least about 15 nucleotides that are complementary to the antisense region.

In one embodiment, the siNA molecule can be designed to target a sequence that is unique to a specific target RNA sequence (e.g., a single allele or single nucleotide polymorphism (SNP)) due to the high degree of specificity that the siNA molecule requires to mediate RNAi activity.

In one embodiment, nucleic acid molecules of the disclosure that act as mediators of the RNA interference gene silencing response are double-stranded nucleic acid molecules. In another embodiment, the siNA molecules of the disclosure consist of duplex nucleic acid molecules containing about 15 to about 30 base pairs between oligonucleotides comprising about 15 to about 30 (e.g., about 15, 16, 17, 18, 19, 20, 21, 22, 23, 24, 25, 26, 27, 28, 29, or 30) nucleotides. In yet another embodiment, siNA molecules of the disclosure comprise duplex nucleic acid molecules with overhanging ends of about 1 to about 3 (e.g., about 1, 2, or 3) nucleotides, for example, about 21-nucleotide duplexes with about 19 base pairs and 3′-terminal mononucleotide, dinucleotide, or trinucleotide overhangs. In yet another embodiment, siNA molecules of the disclosure comprise duplex nucleic acid molecules with blunt ends, where both ends are blunt, or alternatively, where one of the ends is blunt.

Non-limiting examples of encompassed chemical modifications include, without limitation, phosphorothioate internucleotide linkages, deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, (e.g., RNA based siNA constructs), are shown to preserve RNAi activity in cells while at the same time, dramatically increasing the serum stability of these compounds.

In one embodiment, a siNA molecule of the disclosure comprises modified nucleotides while maintaining the ability to mediate RNAi. The modified nucleotides can be used to improve in vitro or in vivo characteristics such as stability, activity, and/or bioavailability. For example, a siNA molecule of the disclosure can comprise modified nucleotides as a percentage of the total number of nucleotides present in the siNA molecule. As such, a siNA molecule of the disclosure can generally comprise about 5% to about 100% modified nucleotides (e.g., about 5%, 10%, 15%, 20%, 25%, 30%, 35%, 40%, 45%, 50%, 55%, 60%, 65%, 70%, 75%, 50%, 85%, 90%, 95% or 100% modified nucleotides). The actual percentage of modified nucleotides present in a given siNA molecule will depend on the total number of nucleotides present in the siNA. If the siNA molecule is single stranded, the percent modification can be based upon the total number of nucleotides present in the single stranded siNA molecules. Likewise, if the siNA molecule is double stranded, the percent modification can be based upon the total number of nucleotides present in the sense strand, antisense strand, or both the sense and antisense strands.

One aspect of the disclosure features a double-stranded short interfering nucleic acid (siNA) molecule that down-regulates expression of a target gene or that directs cleavage of a target RNA. In one embodiment, the double stranded siNA molecule comprises one or more chemical modifications and each strand of the double-stranded siNA is about 21 nucleotides long. In one embodiment, the double-stranded siNA molecule does not contain any ribonucleotides. In another embodiment, the double-stranded siNA molecule comprises one or more ribonucleotides. In one embodiment, each strand of the double-stranded siNA molecule independently comprises about 15 to about 30 (e.g., about 15, 16, 17, 18, 19, 20, 21, 22, 23, 24, 25, 26, 27, 28, 29, or 30) nucleotides, wherein each strand comprises about 15 to about 30 (e.g., about 15, 16, 17, 18, 19, 20, 21, 22, 23, 24, 25, 26, 27, 28, 29, or 30) nucleotides that are complementary to the nucleotides of the other strand. In one embodiment, one of the strands of the double-stranded siNA molecule comprises a nucleotide sequence that is complementary to a nucleotide sequence or a portion thereof of the gene, and the second strand of the double-stranded siNA molecule comprises a nucleotide sequence substantially similar to the nucleotide sequence of the gene or a portion thereof.

In another embodiment, the disclosure features a double-stranded short interfering nucleic acid (siNA) molecule that down-regulates expression of a target gene or that directs cleavage of a target RNA, comprising an antisense region, wherein the antisense region comprises a nucleotide sequence that is complementary to a nucleotide sequence of the gene or a portion thereof, and a sense region, wherein the sense region comprises a nucleotide sequence substantially similar to the nucleotide sequence of the gene or a portion thereof. In one embodiment, the antisense region and the sense region independently comprise about 15 to about 30 (e.g. about 15, 16, 17, 18, 19, 20, 21, 22, 23, 24, 25, 26, 27, 28, 29, or 30) nucleotides, wherein the antisense region comprises about 15 to about 30 (e.g. about 15, 16, 17, 18, 19, 20, 21, 22, 23, 21, 25, 26, 27, 28, 29, or 30) nucleotides that are complementary to nucleotides of the sense region.

In another embodiment, the disclosure features a double-stranded short interfering nucleic acid (siNA) molecule that down-regulates expression of a target gene or that directs cleavage of a target RNA, comprising a sense region and an antisense region, wherein the antisense region comprises a nucleotide sequence that is complementary to a nucleotide sequence of RNA encoded by the gene or a portion thereof and the sense region comprises a nucleotide sequence that is complementary to the antisense region.

In one embodiment, a siNA molecule of the disclosure comprises blunt ends, i.e., ends that do not include any overhanging nucleotides.

In one embodiment, any siNA molecule of the disclosure can comprise one or more blunt ends, i.e. where a blunt end does not have any overhanging nucleotides. In one embodiment, the blunt ended siNA molecule has a number of base pairs equal to the number of nucleotides present in each strand of the siNA molecule. In another embodiment, the siNA molecule comprises one blunt end, for example wherein the 5′-end of the antisense strand and the 3′-end of the sense strand do not have any overhanging nucleotides. In another example, the siNA molecule comprises one blunt end, for example wherein the 3′-end of the antisense strand and the 5′-end of the sense strand do not have any overhanging nucleotides. In another example, a siNA molecule comprises two blunt ends, for example wherein the 3′-end of the antisense strand and the 5′-end of the sense strand as well as the 5′-end of the antisense strand and 3′-end of the sense strand do not have any overhanging nucleotides. A blunt ended siNA molecule can comprise, for example, from about 15 to about 30 nucleotides (e.g., about 15, 16, 17, 18, 19, 20, 21, 22, 23, 24, 25, 26, 27, 28, 29, or 30 nucleotides). Other nucleotides present in a blunt ended siNA molecule can comprise, for example, mismatches, bulges, loops, or wobble base pairs to modulate the activity of the siNA molecule to mediate RNA interference.

By “blunt ends” is meant symmetric termini or termini of a double stranded siNA molecule having no overhanging nucleotides. The two strands of a double stranded siNA molecule align with each other without over-hanging nucleotides at the termini. For example, a blunt ended siNA construct comprises terminal nucleotides that are complementary between the sense and antisense regions of the siNA molecule.

In one embodiment, the disclosure features a double-stranded short interfering nucleic acid (siNA) molecule that down-regulates expression of a target gene or that directs cleavage of a target RNA, wherein the siNA molecule is assembled from two separate oligonucleotide fragments wherein one fragment comprises the sense region and the second fragment comprises the antisense region of the siNA molecule. The sense region can be connected to the antisense region via a linker molecule, such as a polynucleotide linker or a non-nucleotide linker.

In one embodiment, the disclosure features double-stranded short interfering nucleic acid (siNA) molecule that down-regulates expression of a target gene or that directs cleavage of a target RNA, wherein the siNA molecule comprises about 15 to about 30 (e.g. about 15, 16, 17, 18, 19, 20, 21, 22, 23, 24, 25, 26, 27, 28, 29, or 30) base pairs, and wherein each strand of the siNA molecule comprises one or more chemical modifications. In another embodiment, one of the strands of the double-stranded siNA molecule comprises a nucleotide sequence that is complementary to a nucleotide sequence of a gene or a portion thereof, and the second strand of the double-stranded siNA molecule comprises a nucleotide sequence substantially similar to the nucleotide sequence or a portion thereof of the gene. In another embodiment, one of the strands of the double-stranded siNA molecule comprises a nucleotide sequence that is complementary to a nucleotide sequence of a gene or portion thereof, and the second strand of the double-stranded siNA molecule comprises a nucleotide sequence substantially similar to the nucleotide sequence or portion thereof of the gene. In another embodiment, each strand of the siNA molecule comprises about 15 to about 30 (e.g. about 15, 16, 17, 18, 19, 20, 21, 22, 23, 24, 25, 26, 27, 28, 29, or 30) nucleotides, and each strand comprises at least about 15 to about 30 (e.g. about 15, 16, 17, 18, 19, 20, 21, 22, 23, 24, 25, 26, 27, 28, 29, or 30) nucleotides that are complementary to the nucleotides of the other strand.

In one embodiment, a siNA molecule of the disclosure comprises no ribonucleotides. In another embodiment, a siNA molecule of the disclosure comprises ribonucleotides.

In one embodiment, a siNA molecule of the disclosure comprises an antisense region comprising a nucleotide sequence that is complementary to a nucleotide sequence of a target gene or a portion thereof, and the siNA further comprises a sense region comprising a nucleotide sequence substantially similar to the nucleotide sequence of the target gene or a portion thereof. In another embodiment, the antisense region and the sense region each comprise about 15 to about 30 (e.g. about 15, 16, 17, 18, 19, 20, 21, 22, 23, 24, 25, 26, 27, 28, 29, or 30) nucleotides and the antisense region comprises at least about 15 to about 30 (e.g. about 15, 16, 17, 18, 19, 20, 21, 22, 23, 24, 25, 26, 27, 28, 29, or 30) nucleotides that are complementary to nucleotides of the sense region. In another embodiment, the siNA is a double stranded nucleic acid molecule, where each of the two strands of the siNA molecule independently comprise about 15 to about 40 (e.g. about 15, 16, 17, 18, 19, 20, 21, 22, 23, 24, 25, 26, 27, 28, 29, 30, 31, 23, 33, 34, 35, 36, 37, 38, 39, or 40) nucleotides, and where one of the strands of the siNA molecule comprises at least about 15 (e.g. about 15, 16, 17, 18, 19, 20, 21, 22, 23, 24 or 25 or more) nucleotides that are complementary to the nucleic acid sequence of the gene or a portion thereof.

In one embodiment, a siNA molecule of the disclosure comprises a sense region and an antisense region, wherein the antisense region comprises a nucleotide sequence that is complementary to a nucleotide sequence of RNA encoded by a target gene, or a portion thereof, and the sense region comprises a nucleotide sequence that is complementary to the antisense region. In one embodiment, the siNA molecule is assembled from two separate oligonucleotide fragments, wherein one fragment comprises the sense region and the second fragment comprises the antisense region of the siNA molecule. In another embodiment, the sense region is connected to the antisense region via a linker molecule. In another embodiment, the sense region is connected to the antisense region via a linker molecule, such as a nucleotide or non-nucleotide linker.

In one embodiment, the disclosure features a double-stranded short interfering nucleic acid (siNA) molecule that down-regulates expression of a target gene or that directs cleavage of a target RNA comprising a sense region and an antisense region, wherein the antisense region comprises a nucleotide sequence that is complementary to a nucleotide sequence of RNA encoded by the target gene or a portion thereof and the sense region comprises a nucleotide sequence that is complementary to the antisense region, and wherein the siNA molecule has one or more modified pyrimidine and/or purine nucleotides. In one embodiment, the pyrimidine nucleotides in the sense region are 2′-O-methylpyrimidine nucleotides or 2′-deoxy-2′-fluoro pyrimidine nucleotides and the purine nucleotides present in the sense region are 2′-deoxy purine nucleotides. In another embodiment, the pyrimidine nucleotides in the sense region are 2′-deoxy-2′-fluoro pyrimidine nucleotides and the purine nucleotides present in the sense region are 2′-O-methyl purine nucleotides. In another embodiment, the pyrimidine nucleotides in the sense region are 2′-deoxy-2′-fluoro pyrimidine nucleotides and the purine nucleotides present in the sense region are 2′-deoxy purine nucleotides. In one embodiment, the pyrimidine nucleotides in the antisense region are 2′-deoxy-2′-fluoro pyrimidine nucleotides and the purine nucleotides present in the antisense region are 2′-O-methyl or 2′-deoxy purine nucleotides. In another embodiment of any of the above-described siNA molecules, any nucleotides present in a non-complementary region of the sense strand (e.g. overhang region) are 2′-deoxy nucleotides.

In one embodiment, the disclosure features a double-stranded short interfering nucleic acid (siNA) molecule that down-regulates expression of a target gene or that directs cleavage of a target RNA, wherein the siNA molecule is assembled from two separate oligonucleotide fragments wherein one fragment comprises the sense region and the second fragment comprises the antisense region of the siNA molecule, and wherein the fragment comprising the sense region includes a terminal cap moiety at the 5′-end, the 3′-end, or both of the 5′ and 3′ ends of the fragment. In one embodiment, the terminal cap moiety is en inverted deoxy abasic moiety or glyceryl moiety. In one embodiment, each of the two fragments of the siNA molecule independently comprise about 15 to about 30 (e.g. about 15, 16, 17, 18, 19, 20, 21, 22, 23, 24, 25, 26, 27, 28, 29, or 30) nucleotides. In another embodiment, each of the two fragments of the siNA molecule independently comprise about 15 to about 40 (e.g. about 15, 16, 17, 18, 19, 20, 21, 22, 23, 24, 25, 25, 27, 28, 29, 30, 31, 23, 33, 34, 35, 36, 37, 38, 39, or 40) nucleotides. In a non-limiting example, each of the two fragments of the siNA molecule comprise about 21 nucleotides.

In one embodiment, the disclosure features a siNA molecule comprising at least one modified nucleotide, wherein the modified nucleotide is a 2′-deoxy-2′-fluoro nucleotide. The siNA can be, for example, about 15 to about 40 nucleotides in length. In one embodiment, all pyrimidine nucleotides present in the siNA are 2′-deoxy-2′-fluoro pyrimidine nucleotides. In one embodiment, the modified nucleotides in the siNA include at least one 2′-deoxy-2′-fluoro cytidine or 2′-deoxy-2′-fluoro uridine nucleotide. In another embodiment, the modified nucleotides in the siNA include at least one 2′-fluoro cytidine and at least one 2′-deoxy-2′-fluoro uridine nucleotides. In one embodiment, all uridine nucleotides present in the siNA are 2′-deoxy-2′-fluoro uridine nucleotides. In one embodiment, all cytidine nucleotides present in the siNA are 2′-deoxy-2′-fluoro cytidine nucleotides. In one embodiment, all adenosine nucleotides present in the siNA are 2′-deoxy-2′-fluoro adenosine nucleotides. In one embodiment, all guanosine nucleotides present in the siNA are 2′-deoxy-2′-fluoro guanosine nucleotides. The siNA can further comprise at least one modified internucleotidic linkage, such as phosphorothioate linkage. In one embodiment, the 2′-deoxy-2′-fluoronucleotides are present at specifically selected locations in the siNA that are sensitive to cleavage by ribonucleases, such as locations having pyrimidine nucleotides.

In one embodiment, the disclosure features a double-stranded short interfering nucleic acid (siNA) molecule that down-regulates expression of a target gene or that directs cleavage of a target RNA comprising a sense region and an antisense region, wherein the antisense region comprises a nucleotide sequence that is complementary to a nucleotide sequence of RNA encoded by the gene or a portion thereof and the sense region comprises a nucleotide sequence that is complementary to the antisense region, and wherein the purine nucleotides present in the antisense region comprise 2′-deoxy-purine nucleotides. In an alternative embodiment, the purine nucleotides present in the antisense region comprise 2′-O-methyl purine nucleotides. In either of the above embodiments, the antisense region can comprise a phosphorothioate internucleotide linkage at the 3 end of the antisense region. Alternatively, in either of the above embodiments, the antisense region can comprise a glyceryl modification at the 3′ end of the antisense region. In another embodiment of any of the above-described siNA molecules, any nucleotides present in a non-complementary region of the antisense strand (e.g. overhang region) are 2′-deoxy nucleotides.

In one embodiment, the disclosure features a double-stranded short interfering nucleic acid (siNA) molecule that down-regulates expression of a target gene or that directs cleavage of a target RNA, wherein the siNA molecule is assembled from two separate oligonucleotide fragments wherein one fragment comprises the sense region and the second fragment comprises the antisense region of the siNA molecule. In another embodiment, the siNA molecule is a double stranded nucleic acid molecule, where each strand is about 21 nucleotides long and where about 19 nucleotides of each fragment of the siNA molecule are base-paired to the complementary nucleotides of the other fragment of the siNA molecule, wherein at least two 3′ terminal nucleotides of each fragment of the siNA molecule are not base-paired to the nucleotides of the other fragment of the siNA molecule. In another embodiment, the siNA molecule is a double stranded nucleic acid molecule, where each strand is about 19 nucleotide long and where the nucleotides of each fragment of the siNA molecule are base-paired to the complementary nucleotides of the other fragment of the siNA molecule to form at least about 15 (e.g., 15, 16, 17, 18, or 19) base pairs, wherein one or both ends of the siNA molecule are blunt ends. In one embodiment, each of the two 3 terminal nucleotides of each fragment of the siNA molecule is a 2′-deoxy-pyrimidine nucleotide, such as a 2′-deoxy-thymidine. In another embodiment, all nucleotides of each fragment of the siNA molecule are base-paired to the complementary nucleotides of the other fragment of the siNA molecule. In another embodiment, the siNA molecule is a double stranded nucleic acid molecule of about 19 to about 25 base pairs having a sense region and an antisense region, where about 19 nucleotides of the antisense region are base-paired to the nucleotide sequence or a portion thereof of the RNA encoded by the target gene. In another embodiment, about 21 nucleotides of the antisense region are base-paired to the nucleotide sequence or a portion thereof of the RNA encoded by the target gene. In any of the above embodiments, the 5′-end of the fragment comprising said antisense region can optionally include a phosphate group.

In one embodiment, the disclosure features a chemically synthesized double stranded RNA molecule that directs cleavage of a target RNA via RNA interference, wherein each strand of said RNA molecule is about 15 to about 30 nucleotides in length; one strand of the RNA molecule comprises nucleotide sequence having sufficient complementarity to the target RNA for the RNA molecule to direct cleavage of the target RNA via RNA interference; and wherein at least one strand of the RNA molecule optionally comprises one or more chemically modified nucleotides described herein, such as without limitation deoxynucleotides, 2′-O-methyl nucleotides, 2′-deoxy-2′-fluoro nucleotides, 2′-O-methoxyethyl nucleotides etc.

In one embodiment, target RNA of the disclosure comprises non-coding RNA sequence (e.g., miRNA, snRNA siRNA etc.).

In one embodiment, the disclosure features a medicament comprising a siNA molecule of the disclosure.

In one embodiment, the disclosure features an active ingredient comprising a siNA molecule of the disclosure.

In one embodiment, the disclosure features the use of a double-stranded short interfering nucleic acid (siNA) molecule to inhibit, down-regulate, or reduce expression of a RAD9 gene or that directs cleavage of a target RAD9 RNA, wherein the siNA molecule comprises one or more chemical modifications and each strand of the double-stranded siNA is independently about 15 to about 30 or more (e.g., about 15, 16, 17, 18, 19, 20, 21, 22, 23, 24, 25, 26, 27, 28, 29 or 30 or more) nucleotides long. In one embodiment, the siNA molecule of the disclosure is a double stranded nucleic acid molecule comprising one or more chemical modifications, where each of the two fragments of the siNA molecule independently comprise about 15 to about 40 (e.g. about 15, 16, 17, 18, 19, 20, 21, 22, 23, 24, 25, 26, 27, 28, 29, 30, 31, 23, 33, 34, 35, 35, 37, 38, 39, or 40) nucleotides and where one of the strands comprises at least 15 nucleotides that are complementary to nucleotide sequence of target RNA or a portion thereof. In a non-limiting example, each of the two fragments of the siNA molecule comprise about 21 nucleotides. In another embodiment, the siNA molecule is a double stranded nucleic acid molecule comprising one or more chemical modifications, where each strand is about 21 nucleotide long and where about 19 nucleotides of each fragment of the siNA molecule are base-paired to the complementary nucleotides of the other fragment of the siNA molecule, wherein at least two 3′ terminal nucleotides of each fragment of the siNA molecule are not base-paired to the nucleotides of the other fragment of the siNA molecule. In another embodiment, the siNA molecule is a double stranded nucleic acid molecule comprising one or more chemical modifications, where each strand is about 19 nucleotide long and where the nucleotides of each fragment of the siNA molecule are base-paired to the complementary nucleotides of the other fragment of the siNA molecule to form at least about 15 (e.g., 15, 16, 17, 18, or 19) base pairs, wherein one or both ends of the siNA molecule are blunt ends. In one embodiment, each of the two 3′ terminal nucleotides of each fragment of the siNA molecule is a 2′-deoxy-pyrimidine nucleotide, such as a 2′-deoxy-thymidine. In another embodiment, all nucleotides of each fragment of the siNA molecule are base-paired to the complementary nucleotides of the other fragment of the siNA molecule. In another embodiment, the siNA molecule is a double stranded nucleic acid molecule of about 19 to about 25 base pairs having a sense region and an antisense region and comprising one or more chemical modifications, where about 19 nucleotides of the antisense region are base-paired to the nucleotide sequence or a portion thereof of the RNA encoded by the target gene. In another embodiment, about 21 nucleotides of the antisense region are base-paired to the nucleotide sequence or a portion thereof of the RNA encoded by the target gene. In any of the above embodiments, the 5′-end of the fragment comprising said antisense region can optionally include a phosphate group.

In one embodiment, the disclosure features the use of a double-stranded short interfering nucleic acid (siNA) molecule that inhibits, down-regulates, or reduces expression of a target gene or that directs cleavage of a target RNA, wherein one of the strands of the double-stranded siNA molecule is an antisense strand which comprises nucleotide sequence that is complementary to nucleotide sequence of target RNA or a portion thereof, the other strand is a sense strand which comprises nucleotide sequence that is complementary to a nucleotide sequence of the antisense strand and wherein a majority of the pyrimidine nucleotides present in the double-stranded siNA molecule comprises a sugar modification (e.g., 2′-deoxy-2′-fluoro, 2′-O-methyl, or 2′-deoxy modifications).

In one embodiment, the disclosure features a double-stranded short interfering nucleic acid (siNA) molecule that inhibits, down-regulates, or reduces expression of a target gene or that directs cleavage of a target RNA, wherein one of the strands of the double-stranded siNA molecule is an antisense strand which comprises nucleotide sequence that is complementary to nucleotide sequence of target RNA or a portion thereof, wherein the other strand is a sense strand which comprises nucleotide sequence that is complementary to a nucleotide sequence of the antisense strand and wherein a majority of the pyrimidine nucleotides present in the double-stranded siNA molecule comprises a sugar modification (e.g., 2-deoxy-2′-fluoro, 2′-O-methyl, or 2′-deoxy modifications).

In one embodiment, the disclosure features a double-stranded short interfering nucleic acid (siNA) molecule that inhibits, down-regulates, or reduces expression of a gene or that directs cleavage of a target RNA, wherein one of the strands of the double-stranded siNA molecule is an antisense strand which comprises nucleotide sequence that is complementary to nucleotide sequence of target RNA that encodes a protein or portion thereof, the other strand is a sense strand which comprises nucleotide sequence that is complementary to a nucleotide sequence of the antisense strand and wherein a majority of the pyrimidine nucleotides present in the double-stranded siNA molecule comprises a sugar modification. In one embodiment, each strand of the siNA molecule comprises about 15 to about 30 or more (e.g., about 15, 16, 17, 19, 19, 20, 21, 22, 23, 24, 25, 26, 27, 28, 29, or 30 or more nucleotides, wherein each strand comprises at least about 15 nucleotides that are complementary to the nucleotides of the other strand. In one embodiment, the siNA molecule is assembled from two oligonucleotide fragments, wherein one fragment comprises the nucleotide sequence of the antisense strand of the siNA molecule and a second fragment comprises nucleotide sequence of the sense region of the siNA molecule. In one embodiment, the sense strand is connected to the antisense strand via a linker molecule, such as a polynucleotide linker or a non-nucleotide linker. In a further embodiment, the pyrimidine nucleotides present in the sense strand are 2′-deoxy-2′ fluoro pyrimidine nucleotides and the purine nucleotides present in the sense region are 2′-deoxy purine nucleotides. In another embodiment, the pyrimidine nucleotides present in the sense strand are 2′-deoxy-2′ fluoro pyrimidine nucleotides and the purine nucleotides present in the sense region are 2-O-methyl purine nucleotides. In still another embodiment, the pyrimidine nucleotides present in the antisense strand are 2-deoxy-2′-fluoro pyrimidine nucleotides and any purine nucleotides present in the antisense strand are 2′-deoxy purine nucleotides. In another embodiment, the antisense strand comprises one or more 2′-deoxy-2′-fluoro pyrimidine nucleotides and one or more 2′-O-methyl purine nucleotides. In another embodiment, the pyrimidine nucleotides present in the antisense strand are 2′-deoxy-2′-fluoro pyrimidine nucleotides and any purine nucleotides present in the antisense strand are 2-O-methyl purine nucleotides. In a further embodiment the sense strand comprises a 3′-end and a 5′-end, wherein a terminal cap moiety (e.g., an inverted deoxy abasic moiety or inverted deoxy nucleotide moiety such as inverted thymidine) is present at the 5′-end, the 3′-end, or both of the 5′ and 3 ends of the sense strand. In another embodiment, the antisense strand comprises a phosphorothioate internucleotide linkage at the 3′ end of the antisense strand. In another embodiment, the antisense strand comprises a glyceryl modification at the 3′ end. In another embodiment, the 5′-end of the antisense strand optionally includes a phosphate group.

In any of the above-described embodiments of a double-stranded short interfering nucleic acid (siNA) molecule that inhibits expression of a target gene or that directs cleavage of a target RNA, wherein a majority of the pyrimidine nucleotides present in the double-stranded siNA molecule comprises a sugar modification, each of the two strands of the siNA molecule can comprise about 15 to about 30 or more (e.g., about 15, 16, 17, 18, 19, 20, 21, 22, 23, 24, 25, 26, 27, 28, 29, or 30 or more) nucleotides. In one embodiment, about 15 to about 30 or more (e.g., about 15, 16, 17, 18, 19, 20, 21, 22, 23, 24, 25, 26, 27, 28, 29, or 30 or more) nucleotides of each strand of the siNA molecule are base-paired to the complementary nucleotides of the other strand of the siNA molecule. In another embodiment, about 15 to about 30 or more (e.g., about 15, 16, 17, 18, 19, 20, 21, 22, 23, 24, 25, 26, 27, 28, 29, or 30 or more) nucleotides of each strand of the siNA molecule are base-paired to the complementary nucleotides of the other strand of the siNA molecule, wherein at least two 3′ terminal nucleotides of each strand of the siNA molecule are not base-paired to the nucleotides of the other strand of the siNA molecule. In another embodiment, each of the two 3′ terminal nucleotides of each fragment of the siNA molecule is a 2′-deoxy-pyrimidine, such as 2′-deoxy-thymidine. In one embodiment, each strand of the siNA molecule is base-paired to the complementary nucleotides of the other strand of the siNA molecule. In one embodiment, about 15 to about 30 (e.g., about 15, 15, 17, 18, 19, 20, 21, 22, 23, 24, 25, 26, 27, 28, 29, or 30) nucleotides of the antisense strand are base-paired to the nucleotide sequence of the target RNA or a portion thereof. In one embodiment, about 18 to about 25 (e.g., about 18, 19, 20, 21, 22, 23, 24, or 25) nucleotides of the antisense strand are base-paired to the nucleotide sequence of the target RNA or a portion thereof.

In one embodiment, the disclosure features a double-stranded short interfering nucleic acid (siNA) molecule that inhibits expression of a target gene or that directs cleavage of a target RNA, wherein one of the strands of the double-stranded siNA molecule is an antisense strand which comprises nucleotide sequence that is complementary to nucleotide sequence of target RNA or a portion thereof, the other strand is a sense strand which comprises nucleotide sequence that is complementary to a nucleotide sequence of the antisense strand and wherein a majority of the pyrimidine nucleotides present in the double-stranded siNA molecule comprises a sugar modification, and wherein the 5′-end of the antisense strand optionally includes a phosphate group.

In one embodiment, the disclosure features a double-stranded short interfering nucleic acid (siNA) molecule that inhibits expression of a target gene or that directs cleavage of a target RNA, wherein one of the strands of the double-stranded siNA molecule is an antisense strand which comprises nucleotide sequence that is complementary to nucleotide sequence of target RNA or a portion thereof, the other strand is a sense strand which comprises nucleotide sequence that is complementary to a nucleotide sequence of the antisense strand and wherein a majority of the pyrimidine nucleotides present in the double-stranded siNA molecule comprises a sugar modification, and wherein the nucleotide sequence or a portion thereof of the antisense strand is complementary to a nucleotide sequence of the untranslated region or a portion thereof of the target RNA.

In one embodiment, the disclosure features a double-stranded short interfering nucleic acid (siNA) molecule that inhibits expression of a target gene or that directs cleavage of a target RNA, wherein one of the strands of the double-stranded siNA molecule is an antisense strand which comprises nucleotide sequence that is complementary to nucleotide sequence of target RNA or a portion thereof, wherein the other strand is a sense strand which comprises nucleotide sequence that is complementary to a nucleotide sequence of the antisense strand, wherein a majority of the pyrimidine nucleotides present in the double-stranded siNA molecule comprises a sugar modification, and wherein the nucleotide sequence of the antisense strand is complementary to a nucleotide sequence of the target RNA or a portion thereof that is present in the target RNA.

In one embodiment, the disclosure features a composition comprising a siNA molecule of the disclosure in a pharmaceutically acceptable carrier or diluent.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

In any of the embodiments of siNA molecules described herein, the antisense region of a siNA molecule of the disclosure can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of a siNA molecule of the disclosure can comprise ribonucleotides or deoxyribonucleotides that are chemically-modified at a nucleic acid sugar, base, or backbone. In any of the embodiments of siNA molecules described herein, the terminal nucleotide overhangs can comprise one or more universal base ribonucleotides. In any of the embodiments of siNA molecules described herein, the 3′-terminal nucleotide overhangs can comprise one or more acyclic nucleotides.

One embodiment of the disclosure provides an expression vector comprising a nucleic acid sequence encoding at least one siNA molecule of the disclosure in a manner that allows expression of the nucleic acid molecule. Another embodiment of the disclosure provides a mammalian cell comprising such an expression vector. The mammalian cell can be a human cell. The siNA molecule of the expression vector can comprise a sense region and an antisense region. The antisense region can comprise sequence complementary to a RNA or DNA sequence encoding the target and the sense region can comprise sequence complementary to the antisense region. The siNA molecule can comprise two distinct strands having complementary sense and antisense regions. The siNA molecule can comprise a single strand having complementary sense and antisense regions.

In one embodiment, the disclosure features a chemically-modified short interfering nucleic acid (siNA) molecule capable of mediating RNA interference (RNAi) against a target polynucleotide (e.g., DNA or RNA) inside a cell or reconstituted in vitro system, wherein the chemical modification comprises one or more phosphorothioate internucleotide linkages. For example, in a non-limiting example, the disclosure features a chemically-modified short interfering nucleic acid (siNA) having about 1, 2, 3, 4, 5, 6, 7, 8 or more phosphorothioate internucleotide linkages in one siNA strand. In yet another embodiment, the disclosure features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the disclosure can comprise one or more phosphorothioate internucleotide linkages at the 3′-end, the 5′-end, or both of the 3′- and 5′-ends of the sense strand, the antisense strand, or both strands. For example, an exemplary siNA molecule of the disclosure can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of the disclosure can comprise one or more (e.g., about 1, 2, 3, 4, 5, 5, 7, 8, 9, 10, or more) pyrimidine phosphorothioate internucleotide linkages in the sense strand, the antisense strand, or both strands. In yet another non-limiting example, an exemplary siNA molecule of the disclosure can comprise one or more (e.g., about 1, 2, 3, 4, 5, 6, 7, 3, 9, 10, or more) purine phosphorothioate internucleotide linkages in the sense strand, the antisense strand, or both strands.

In one embodiment, a siNA molecule of the disclosure is featured, wherein the sense strand comprises one or more, for example, about 1, 2, 3, 4, 5, 6, 7, 8, 9, 10, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 10 or more, specifically about 1, 2, 3, 4, 5, 6, 7, 8, 9, 10, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10, or more, pyrimidine nucleotides of the sense and/or antisense siNA strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate internucleotide linkages and/or a terminal cap molecule at the 3′-end, the 5′-end, or both of the 3′- and 5′-ends, being present in the same or different strand.

In another embodiment, a siNA molecule of the disclosure is featured, wherein the sense strand comprises about 1 to about 5, specifically about 1, 2, 3, 4, or 5 phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base modified nucleotides, and optionally a terminal cap molecule at the 3-end, the 5′-end, or both of the 3′- and 5′-ends of the sense strand; and wherein the antisense strand comprises about 1 to about 5 or more, specifically about 1, 2, 3, 4, 5, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or mere, for example about 1, 2, 3, 4, 5, 6, 7, 8, 9, 10, or more, pyrimidine nucleotides of the sense and/or antisense siNA strand are chemically-modified with 2′-deoxy, 2′-O-methyl and/or 2′-deoxy-2′-fluoro nucleotides, with or without about 1 to about 5 or more, for example about 1, 2, 3, 4, 5, or more phosphorothioate internucleotide linkages and/or a terminal cap molecule at the 3′-end, the 5′-end, or both of the 3′- and 5′-ends, being present in the same or different strand.

In one embodiment, a siNA molecule of the disclosure is featured, wherein the antisense strand comprises one or more, for example, about 1, 2, 3, 4, 5, 6, 7, 8, 9, 10, or more phosphorothioate internucleotide linkages, and/or about one or more (e.g., about 1, 2, 3, 4, 5, 6, 7, 6, 9, or more) 2′-deoxy, 2′-O-methyl, 2′-deoxy-2′-fluoro, and/or one or more (e.g., about 1, 2, 3, 4, 5, 6, 7, 6, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 10 or more, specifically about 1, 2, 3, 4, 5, 5, 7, 6, 9, 10 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense siNA strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate internucleotide linkages and/or a terminal cap molecule at the 3′-end, the 5′-end, or both of the 3′ and 5′-ends, being present in the same or different strand.

In another embodiment, a siNA molecule of the disclosure is featured, wherein the antisense strand comprises about 1 to about 5 or more, specifically about 1, 2, 3, 4, 5 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 5 or more, specifically about 1, 2, 3, 4, 5 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In another embodiment, one or more, for example about 1, 2, 3, 4, 5, 6, 7, 8, 9, 10 or more pyrimidine nucleotides of the sense and/or antisense siNA strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In one embodiment, a chemically-modified short interfering nucleic acid (siNA) molecule of the disclosure comprises about 1 to about 5 or more (specifically about 1, 2, 3, 4, 5 or more) phosphorothioate internucleotide linkages in each strand of the siNA molecule.

In another embodiment, a siNA molecule of the disclosure comprises 2′-5′ internucleotide linkages. The 2′-5′ internucleotide linkage(s) can be at the 3′-end, the 5′-end, or both of the 3′- and 5′-ends of one or both siNA sequence strands. In addition, the 2′-5′ internucleotide linkage(s) can be present at various other positions within one or both siNA sequence strands, for example, about 1, 2, 3, 4, 5, 6, 7, 8, 9, 10, or more including every internucleotide linkage of a pyrimidine nucleotide in one or both strands of the siNA molecule can comprise a 2′-5′ internucleotide linkage, or about 1, 2, 3, 4, 5, 6, 7, 8, 9, 10, or more including every internucleotide linkage of a purine nucleotide in one or both strands of the siNA molecule can comprise a 2′-5 internucleotide linkage.

In one embodiment the nucleic acid is an siRNA duplex composed of 21-nt sense and 21-nt antisense strands, paired in a manner to have a 2-nt 3′ overhang. In one embodiment, the 2-nt 3′ overhang comprises 2′-deoxynucleotides.

The nucleic acids can be delivered/administered via a transfection reagent such as Oligofectamine™ (product number: 12252011 from Invitrogen, CA). Oligofectamine has the advantage of being non-toxic to cells. siRNA transfection is also possible by using TransIT-TKO: small interfering RNA (siRNA) Transfection Reagent, which is provided by mirus; jetSI™ made by Polyplus, France, siIMPORTER™, made by Upstate, Mass. Other methods are described in the experimental results section below.

Non-limiting examples of siRNA carriers include those set forth in Ge Q., Filip L., Bai A., Nguyen T., Eisen H. N and Chen J., PNAS 101: 8676-8681 (2004); Urban-Klein B., Werth S., Abuharbeid S., Czubayko F. and Aigner A. Gene Therapy 12: 461-466 (2005) and Hassani Z., Lemkine G.-F., Erbacher P., Alfama G., Giovannangeli C., Behr J.-P., and Demeneix B.-A, J. Gene Med., 7, 198-207 (2005). Examples include linear polyethylenimine, with an ion chloride and water such as jetPEI™.

A nucleic acid of the disclosure may be delivered via a vector so as to effect transcription of the DNA inserted into the vector into a short hairpin RNA or transcription into a complementary sense and an antisense strand which subsequently hybridize to form a siRNA. The latter may be achieved by a vector insert which comprises a promoter sequence/sense strand encoding sequence/termination sequence/spacer sequence/promoter sequence/antisense strand encoding sequence/termination sequence or a promoter sequence/antisense strand encoding sequence/termination sequence/spacer sequence/promoter sequence/sense strand encoding sequence/termination sequence (e.g. see Tuschl, Expanding small RNA interference, Nature Biotechnology, 20:446-448 (2002) hereby incorporated by reference). Promoters include RNA II polymerase promoters, e.g. U6 or H1.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All combinations of the various elements disclosed herein are within the scope of the disclosure.

A RAD9 and human homolog are described in U.S. Pat. No. 5,382,862, which is hereby incorporated by reference.

All combinations of the various elements disclosed herein are within the scope of the invention.

This disclosure will be better understood by reference to the Experimental Details which follow, but those skilled in the art will readily appreciate that the specific experiments detailed are only illustrative of the disclosure as described more fully in the claims which follow thereafter.

### Experimental Details

The molecular underpinnings of prostate disease are not completely defined. Several genes have been implicated in the disease process, including PTEN (1), c-myc (2), and IGF-1 (3). Regulation of androgen receptor by genes such as Wnt1 is also likely to impact on prostate cancer pathogenesis (4), Mouse models have contributed to understanding the impact of genotype on prostate cancer development (for review see 5, 6), A few studies have found associations between certain chromosome aberrations and prostate cancer. Loss of chromosome 8p23.2 is associated with advanced Stage disease, and gain in 11q13.1 is linked to Stage and Grade independent postoperative recurrence (7).

Rad9 is an evolutionarily conserved gene found in yeasts and mammals. It regulates basic cellular processes, including DNA damage induced cell cycle checkpoints, apoptosis and the maintenance of genomic stability. These functions are often associated with genes that impact on carcinogenesis, such as tumor suppressors or oncogenes. Here, the link between Rad9 expression and prostate cancer was examined to assess whether there is a functional relationship.

HRAD9 is an evolutionarily conserved human gene first identified as a genetic element important for promoting resistance to DNA damage and regulating cell cycle checkpoints (8). Subsequent analyses indicated that it had a much broader range of activities (for review see 9). The encoded protein can induce apoptosis (10), and regulate genomic stability (11). It has 3′ to 5′ exonuclease activity (12), can bind p53 consensus DNA binding sequences and upregulate transcription of p21 as well as other downstream genes (13, 14, 15), is able to stimulate the carbamoyl phosphate synthetase activity of CAD protein (16), required for de novo synthesis of pyrimidine nucleotides and cell growth, and can bind and stimulate activity of several DNA repair proteins involved primarily in base excision repair (17, 16, 19, 20, 21, 22, 23). It also helps stabilize telomeres, and participates in recombinational repair of damaged DNA (24). Moreover, at least related mouse Mrad9 is critical for embryogenesis (11). Human Rad9 protein has also been linked to androgen receptor (AR). AR is critical for differentiation, growth and maintenance of the prostate. AR can bind androgen (i.e., testosterone or the more active form DHT), whereupon the receptor undergoes a conformational change, moves from cytoplasm to nucleus, and transcriptionally activates genes containing androgen responsive consensus sequences. AR can bind human Rad9 (25, 26). Moreover, the binding represses androgen-induced AR transcription activity in prostate cancer cells, thus altering prostate function.

Herein the relationship between prostate cancer and human Rad9 expression was investigated, based primarily on functions of Rad9 protein in maintaining genomic integrity and on its ability to regulate androgen receptor transactivity. It is disclosed that prostate cancer cells express Rad9 at aberrantly high levels, and such overexpression can be due at least in part to abnormal methylation or gene amplification. It was also found that the degree to which siRNA reduces Rad9 protein levels correlates with the extent of decrease in tumorigenicity of these cells after injection into nude mice. The ability of Rad9 to bind to AR and alter its transactivity was found not universally relevant to prostate cancer though since there was no correlation in the prostate cancer cell lines tested with respect to levels of Rad9, levels of AR, expression of PSA, a downstream target of AR transactivity, and the ability of the cells to form tumors in nude mice. High Rad9 protein levels were detected in 153 of 339 human prostate adenocarcinomas, but just low abundance in 2 of 52 normal prostate tissue controls. Relatively higher levels of Rad9 protein were in general associated with more advanced disease. The results indicate that Rad9 plays a functional role in prostate carcinogenesis and is a biomarker for advanced prostate cancer, as well as a target for therapeutic intervention.

### Results

TABLE 1
Immunohistochemical Staining for Rad9 Protein in Normal and Cancer
Prostate Tissue

| | No. of | Stain | Stain | Stain | Stain | Positive |
|---|---|---|---|---|---|---|
| Cancer Stage | Cases | 0* | +* | ++* | +++* | Staining (%) |
| Normal | 52 | 50 | 2 | | | 3.8% |
| I | 43 | 33 | 10 | | | 23.3% |
| II | 65 | 38 | 23 | 4 | | 41.5% |
| III | 155 | 79 | 52 | 24 | | 49% |
| IV | 72 | 36 | 25 | 8 | 3 | 50% |
| Metastasis | 4 | 0 | 2 | 0 | 2 | 100% |
| Prostate | 339 | | | | | |
| Cancer | | | | | | |
| Cases | | | | | | |
| Positive | 153 | | | | | 45.1% |
| Cases | | | | | | |

*Indicates degree of human HRA D9 protein positive staining: 0, undetectable; +, weak posttive; ++, strong positive; +++, very intense staining. See FIG. 4 for examples.

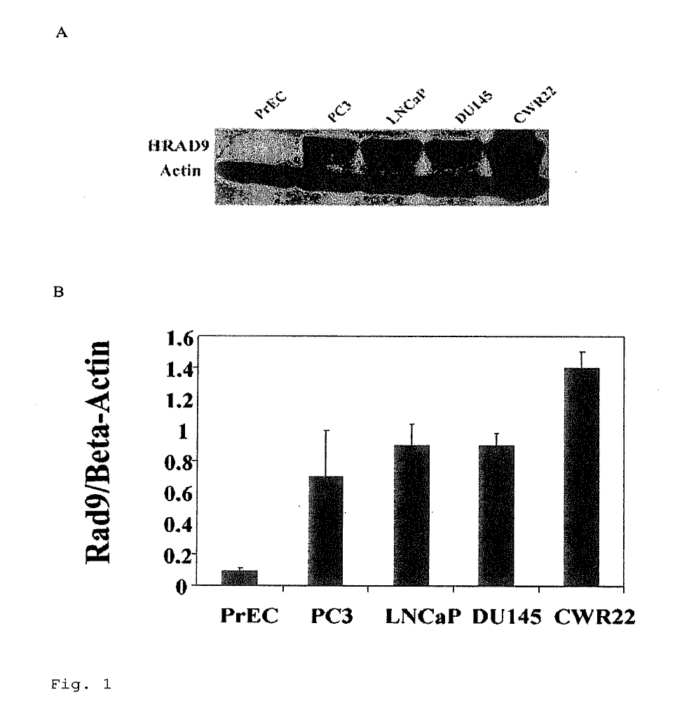

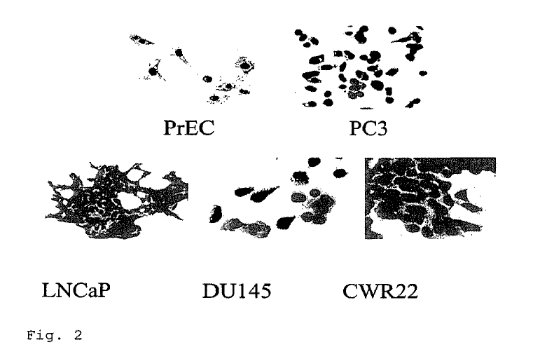

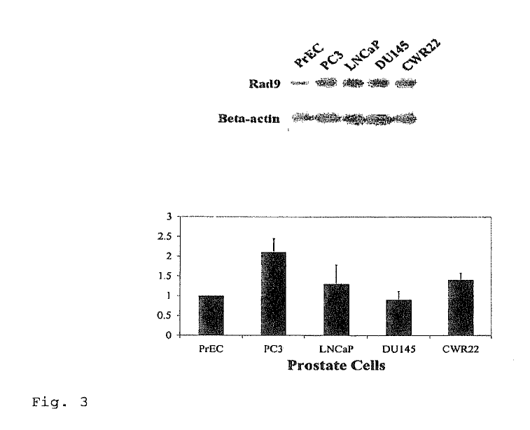

Rad9 RNA and encoded protein levels are high in prostate cancer cells. To assess the relationship between Rad9 and prostate cancer, the level of the encoded protein was examined in four human prostate cancer cell lines, CWR22, DU145, LNCaP and PC-3, as well as in PrEC normal prostate epithelial cells. Western analyses indicate that Rad9 protein is highly abundant in all the cancer cells, relative to PrEC (FIG. 1A). Densitometric analyses of Rad9 to beta-actin (internal control) band intensity ratios for each sample performed three times and averaged indicate an increase in Rad9 protein ranging from 7.8 to 15.5 fold in the prostate cancer versus noncancer cells (FIG. 1B; specific increases were: PC-3, 7.8 fold; LNCaP, 10 fold; PU145, 10 fold; and CWR22, 15.5 fold). Immunohistochemical studies confirm these results (FIG. 2).

Rad9 CpG islands are hypermethylated in DU145 cells. Methylation of CpG islands can control gene expression (31, 32). Cheng and coworkers (27) reported that CpG islands within the second intron of Rad9 might have transcription repressor activity, and methylation of cytosines at those sites neutralize this function, resulting in high expression of Rad9. Methylation status of the promoter was examined, as well as the first and second exon/intron regions of Rad9 in normal and cancer prostate cells to assess whether aberrant methylation might be responsible for high levels of Rad9 expression detected. Bisulfite sequencing was used to identify methylation in 10 independent clones derived from each cell population of interest. CpG islands within Rad9 exons and introns of the prostate normal and cancer cells were methylated, but DU145 cells were relatively hypermethylated (FIG. 4). Excessive methylation was reflected in number of independent clones bearing CpG island methylation (all 10 studied), and number of methylated sites per clone. For example, one clone (number 10) contained 10 methylated CpG islands. Hypermethylation in DU145 was confined to CpG sites within the second Rad9 intron, where the transcription suppressor is located (27). A similar study of the Rad9 promoter region did not detect any CpG island methylation (data not shown). Bisulfite sequencing also yields DNA sequence information, and no mutation in the promoter or first two exons/introns of Rad9 in any of these cells was found.

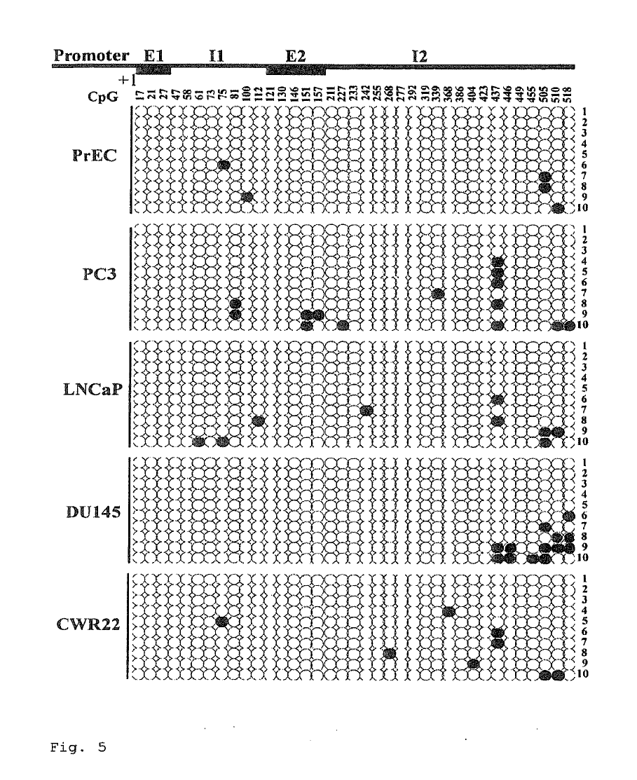

To determine the functional significance of the CpG island methylation, all four cancer and the control cell populations were treated with the demethylating agent 5′-aza-2′-deoxycytidine (0.25 mM). After treatment, the most dramatic reduction in methylation occurred in DU145 cells (compare FIGS. 4 and 5), where baseline starting levels were the highest. For this population, before treatment 10 of 10 clones contained methylated CpG islands, whereas after treatment only 5 of 10 contained methylated sites and each clone had a reduced number of such sites.

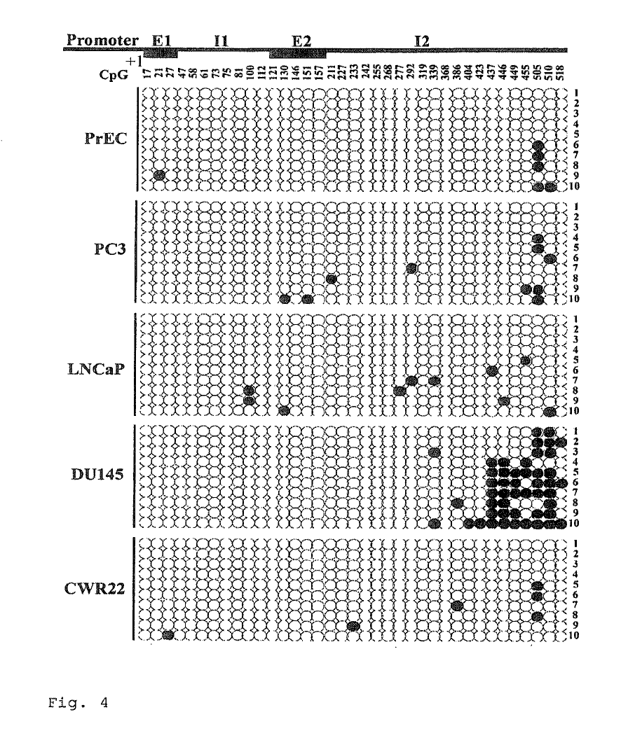

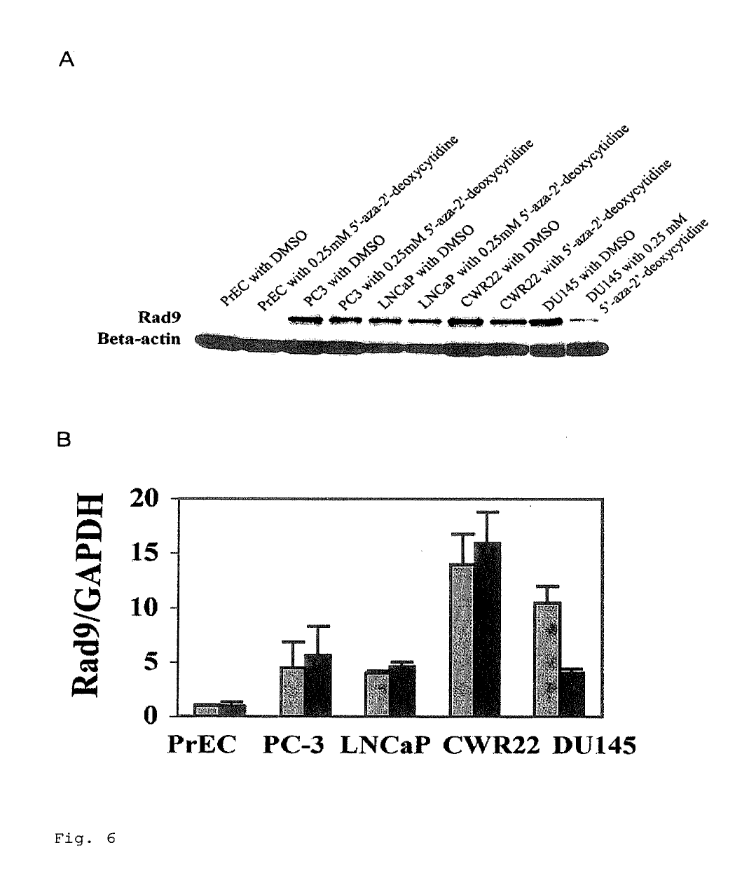

The effects of 5′-aza-2′-deoxycytidine on Rad9 levels were assessed. This chemical dramatically reduced Rad9 protein abundance in DU145 cells (FIG. 6A), suggesting that the high level of the protein in the same cells untreated is due to methylation of CpG islands. Similar treatment of the other cell populations did not reduce Rad9 abundance. Use of 5′-aza-2′-deoxycytidine at concentrations above 0.25 mM (up to 1.0 mM) did not alter Rad9 protein levels any differently than when cells were exposed to the 0.25 mM concentration, and the higher levels of the chemical caused cell death in all populations. Quantitative RT-PCR (FIG. 6B) showed that Rad9 RNA abundance was commensurate with the encoded protein levels in untreated (FIG. 1A, 1B, 2), mock-treated (FIG. 3A) and 5′-aza-2′-deoxycytidine-treated (FIG. 6) cells.

Rad9 gene is amplified in PC-3 cells. Southern blotting was used to determine Rad9 copy number in the four prostate cancer and PrEC noncancerous cells. DNA from these cells was probed with Rad9 and the beta-actin gene (internal control) to assess relative band intensities, which reflect copy number. FIG. 3 represents the average results for three independent Southern blots, and shows the Rad9/beta-actin band intensity ratios, as determined by densitometric analyses. PC-3 has about twice the amount of Rad9 as the other cells, suggesting that gene amplification might, at least in part, be responsible for high levels of the RNA and encoded protein observed. Karyotype analyses of PC-3 cells indicated that there was no selective retention of duplicated chromosome 11, where Rad9 resides, or selective reduction in chromosome 7, where the beta-actin gene is located. Therefore, Rad9 and net the entire chromosome 11 within which it is embedded is increased in copy number in PC-3 cells.

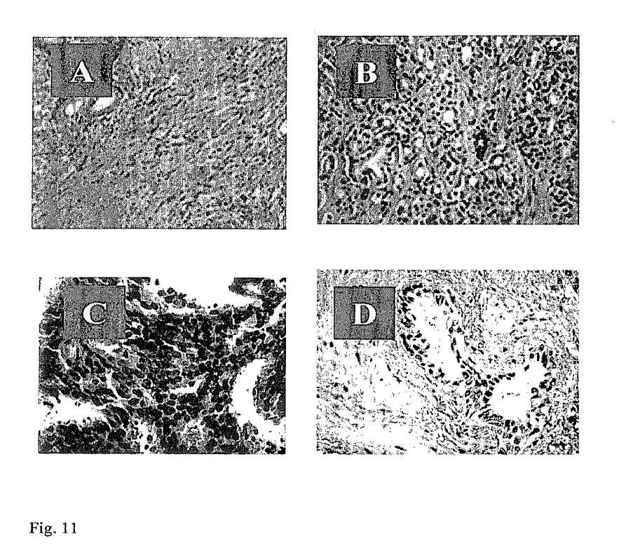

Rad9 protein levels are high in prostate cancer tissues. To extend the findings in prostate cancer cells, primary biopsy material from normal or cancerous prostates was examined immunohistochemically for Rad9 protein. Of 339 prostate cancer samples tested, 153 were positive for HRAD9 (45.19). Of those containing HRAD9, the most intense stain occurred in Stage III and IV adenocarcinomas (and two independent metastases), and all Stage I as well as some Stage 11 positive samples showed a less intense but clearly detectable signal. In contrast, 2 of 52 normal prostate tissues had just weak HRAD9 protein signals (3.8%). Table 1 summarizes the results. FIG. 11 shows what was considered negative (O), weak (+), strong (++), and very intense (+++) HRAD9 protein staining. These data are consistent with the cell culture results and indicate that high levels of HRAD9 are linked to prostate cancer. The statistical significance for relationships between HRAD9 positive staining and cancer, and also stain intensity versus Stage were tested. Groups, as per Table 1, for consideration were simplified, The ++ and +++ staining numbers were combined as they clearly differ from the 0 and + groups, and few are +++. The metastasis group was excluded from calculations since these are not primary tumors. A p-value of <0.001 was then obtained when comparing percent positive by cancer Stage or staining intensity by cancer Stage. p-values are also <0.001 if Stages III and IV are combined. Interestingly, the latter two Stages, unlike I and II, involve metastatic disease. Although information on cancer grade was much more limited, there was a good correlation between severity of grade and levels of Rad9 protein (data not shown).

siRNA

RNA interference (“RNAi”) is a method of post-transcriptional gene regulation that is conserved throughout many eukaryotic organisms. RNAi is induced by short (i.e., <30 nucleotide) double stranded RNA (“dsRNA”) molecules which are present in the cell (Fire, A. et al. (1993), Nature 391: 806-811). These short dsRNA molecules, called “short interfering RNA” or “siRNA,” cause the destruction of messenger RNAs (“mRNAs”) which share sequence homology with the siRNA to within one nucleotide resolution (Elbashir, S. M. et al. (2001), Genes Dev, 15: 108-200). It is believed that the siRNA and the targeted mRNA bind to an “RNA-induced silencing complex” or “RISC”, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of an mRNA is therefore more effective than currently available technologies for inhibiting expression of a target gene.

Elbashir, S. M. et al. (2001), supra, has shown that synthetic siRNA of 21 and 22 nucleotides in length, and which have short 3′ overhangs, are able to induce RNAi of target mRNA in a Drosophila cell lysate. Cultured mammalian cells also exhibit RNAi degradation with synthetic siRNA (Elbashir, S. M. et al. (2001) Nature, 411: 494-498), and RNAi degradation induced by synthetic siRNA has recently been shown in living mice (McCaffrey, A. P. et al. (2002), Nature, 418: 38-39: Xia, H. et al. (2002), Nat. Biotech., 20: 1006-1010). The therapeutic potential of siRNA-induced RNAi degradation has been demonstrated in several recent in vitro studies, including the siRNA-directed inhibition of HIV-1 infection (Novina, C. D. et al. (2002), Nat. Med. 8: 681-686) and reduction of neurotoxic polyglutamine disease protein expression (Xia, H. et al. (2002), supra). In an embodiment the siRNA employed herein to inhibit RAD9 expression is 21 or 22 nucleotides in length. Methods and compositions for gene silencing techniques are described in U.S. Pat. Nos. 6,573,099; 6,506,599; 7,109,165; 7,022,828; 6,995,259; 6,617,438; 6,673,611; 6,849,726; and 6,918,447, which are hereby incorporated by reference.

Post-transcriptional gene silencing techniques are described in U.S. Patent Application No. 20070042983, hereby incorporated by reference. As described therein, RNA interference refers to the process of sequence-specific post-transcriptional gene silencing in animals mediated by short interfering RNAs (siRNAs) (Zamore at al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). The corresponding process in plants (Heifetz et al., International PCT Publication No. NO 99/61631) is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 108). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1999, Nature, 391, 805, were the first to observe RNAi in C. elegans. Bahramian and Zarbl, 1999, Molecular and Cellular Biology, 19, 274-283 and Wianny and Goetz, 1999, Nature Cell Biol., 2, 70, describe RNAi mediated by dsRNA in mammalian systems. Hammond et al., 2000, Nature, 404, 293, describe RNAi in Drosophila cells transfected with dsRNA. Elbashir et al., 2001, Nature, 411, 494 and Tuschl et al., International ECT Publication No. WO 01/75164, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 5877 and Tuschl at al., International PCT Publication No, WO 01/75164) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two-nucleotide 3′-overhangs with deoxyribonucleotides does not have an adverse effect on RNAi activity. Replacing up to four nucleotides on each end of the siRNA with deoxyribonucleotides has been reported to be well tolerated, whereas complete substitution with deoxyribonucleotides results in no RNAi activity (Elbashir et al., 2001, EMBO J., 20, 6677 and Tuschl et al., International ROT Publication No. WO 01/75154). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No, WO 01/58836 preliminarily suggest that siRNA may include modifications to either the phosphate-sugar backbone or the nucleoside to include at least one of a nitrogen or sulfur heteroatom, however, neither application postulates to what extent such modifications would be tolerated in siRNA molecules, nor provides any further guidance or examples of such modified siRNA. Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-O or 4′-C methylene bridge. However, Kreutzer et al. similarly fails to provide examples or guidance as to what extent these modifications would be tolerated in dsRNA molecules.

Parrish et al., 2000, Molecular Cell, 6, 1077-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081. The authors also tested certain modifications at the 2′-position of the nucleotide sugar in the long SiRNA transcripts and found that substituting deoxynucleotides for ribonucleotides produced a substantial decrease in interference activity, especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl)uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 3-iodouracil and 3-(aminoallyl)uracil in the antisense strand resulted in a substantial decrease in RNAi activity as well.

The use of longer dsRNA has been described. For example, Beach et al., International PCT Publication No. WO 01/68836, describes specific methods for attenuating gene expression using endogenously-derived dsRNA. Tuschl et al., International PCT Publication No, WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due to the danger of activating interferon response. Li et al., International PCT Publication No. WO 00/44914, describe the use of specific long (141 bp-488 bp) enzymatically synthesized or vector expressed dsRNAs for attenuating the expression of certain target genes. Zernicka-Goetz et al., International PCT Publication No. WO 01/36646, describe certain methods for inhibiting the expression of particular genes in mammalian cells using certain long (550 bp-714 bp), enzymatically synthesized or vector expressed dsRNA molecules. Fire et al., International PCT Publication No. WO 99/32619, describe particular methods for introducing certain long dsRNA molecules into cells for use in inhibiting gene expression in nematodes. Plaetinck et al., International POT Publication No. WO 00/01846, describe certain methods for identifying specific genes responsible for conferring a particular phenotype in a cell using specific long dsRNA molecules. Mello et al., International PCT Publication No. WO 01/29058, describe the identification of specific genes involved in dsRNA-mediated RNAi. Pachuck et al., International PCT Publication No. WO 00/63364, describe certain long (at least 200 nucleotide) dsRNA constructs. Deschamps Depaillette et al., International PCT Publication No, WO 99/07409, describe specific compositions consisting of particular dsRNA molecules combined with certain anti-viral agents. Waterhouse et al., International PCT Publication No. 99/53050 and 1998, PNAS, 95, 13959-13964, describe certain methods for decreasing the phenotypic expression of a nucleic acid in plant cells using certain dsRNAs, Driscoll et al., International PCT Publication No. WO 01/49844, describe specific DNA expression constructs for use in facilitating gene silencing in targeted organisms.

Others have reported on various RNAi and gene-silencing systems. For example, Parrish et al., 2000, Molecular cell, 6, 1077-1087, describe specific chemically-modified dsRNA constructs targeting the unc-22 gene of C. elegans. Grossniklaus, International PCT Publication No. WO 01/36551, describes certain methods for regulating polycomb gene expression in plants using certain dsRNAs. Churikov et al, International PCT Publication No. WO 01/42443, describe certain methods for modifying genetic characteristics of an organism using certain dsRNAs. Cogoni et al, International POT Publication No. WO 01/53475, describe certain methods for isolating a Neurospora silencing gene and uses thereof. Reed et al., International POT Publication No. WO 01/68836, describe certain methods for gene silencing in plants. Honer at al., International PCT Publication No. WO 01/70944, describe certain methods of drug screening using transgenic nematodes as Parkinson's Disease models using certain dsRNAs. Deak et al., International PCT Publication No. WO 01/72774, describe certain Drosophila-derived gene products that may be related to RNAi in Drosophila. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313, describe certain methods and compositions for inhibiting the function of certain polynucleotide sequences using certain long (over 250 bp), vector expressed dsRNAs. Echeverri et al., International PCT Publication No. WO 02/38805, describe certain C. elegans genes identified via RNAi. Kreutzer at al., International PCT Publications Nos. WO 02/055692, WO 02/055693, and EP 1144623 B1 describes certain methods for inhibiting gene expression using dsRNA. Graham at al., International PCT Publications Nos, WO 99/49029 and WO 01/70949, and AU 4037501 describe certain vector expressed siRNA molecules. Fire et al., U.S. Pat. No. 6,506,559, describe certain methods for inhibiting gene expression in vitro using certain long dsRNA (299 bp-1033 bp) constructs that mediate RNAi. Martinez et al., 2002, Cell, 110, 563-574, describe certain single stranded siRNA constructs, including certain 5′-phosphorylated single stranded siRNAs that mediate RNA interference in Hela cells. Harborth et al., 2003, Antisense & Nucleic Acid Drug Development, 13, 83-105, describe certain chemically and structurally modified siRNA molecules. Chiu and Rana, 2003, RNA, 9, 1034-1048, describe certain chemically and structurally modified siRNA molecules. Woolf et al., International PCT Publication Nos. WO 03/064626 and WO 03/064625 describe certain chemically modified dsRNA constructs.

siRNA-Mediated Decrease in Rad9 Protein Levels Reduces Tumorigenicity of Human Prostate Cancer Cells.

To see if there was more than a simple association between high HRAD9 protein levels and prostate cancer, e.g. whether HRAD9 played a functional role in the disease, further experiments were performed. A mouse tumor model system was used (33). The strategy involved knocking down HRAD9 levels in prostate tumor cells using siRNA, and testing whether that would reduce or eliminate tumor formation post-injection into nude mice.

Three human prostate cancer cell lines, CWR22, DU145 and PC-3, as well as noncancerous prostate PrEC cells were injected at 6×106 cells per site subcutis into backs of nude mice (Harlan Sprague Dawley, Inc., Nu/Nu male, 4 weeks of age) at 8-9 independent sites per cell population. Matrigel is required for LNCaP cells to form tumors in nude mice since they are not as aggressive as the other three populations, CWR22, DU-145 and PC-3. Therefore, the LNCaP cell line was not pursued for the tumor-related experiments. Two sites per animal were usually used to reduce the number of mice needed. Pilot tests showed that number of injection sites per animal, from one up to four did not influence tumor formation frequency or growth. After 2-3 weeks, CWR22, DU145 and PC-3 cells formed tumors at each injection site, but PrEC did not. Tumors were of human origin since they stained positive for human epithelial cell markers (cytokeratin 5, 18, 19; Sigma). To test the significance of high Rad9 protein levels with respect to tumorigenicity, CWR22, DU145 and PC-3 cancer cells were stably transfected with insertless pSUPER.retro.puro or one bearing HRAD9 siRNA.

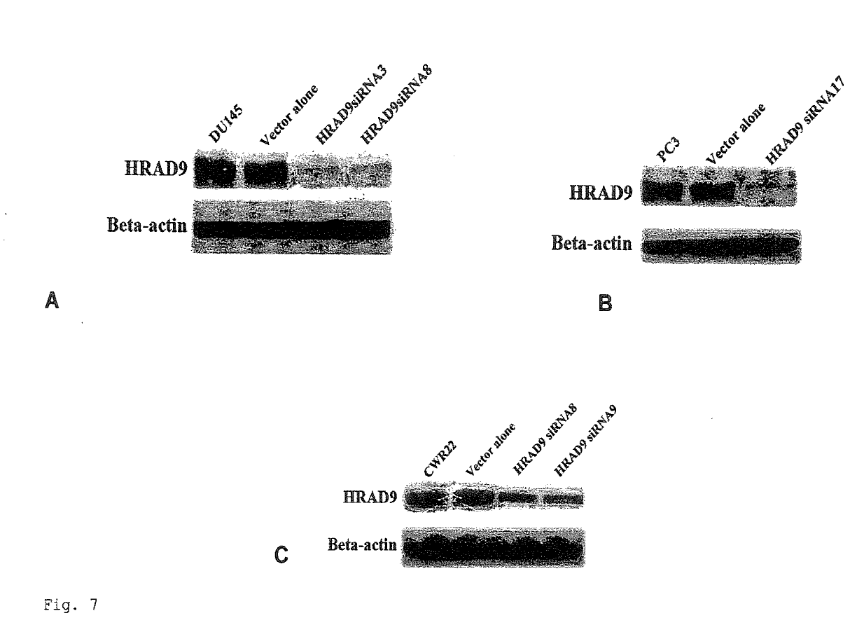

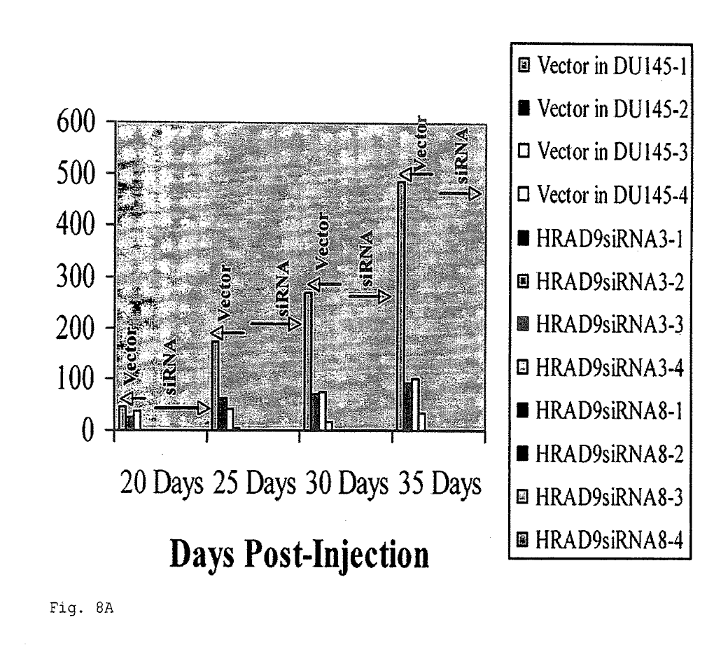

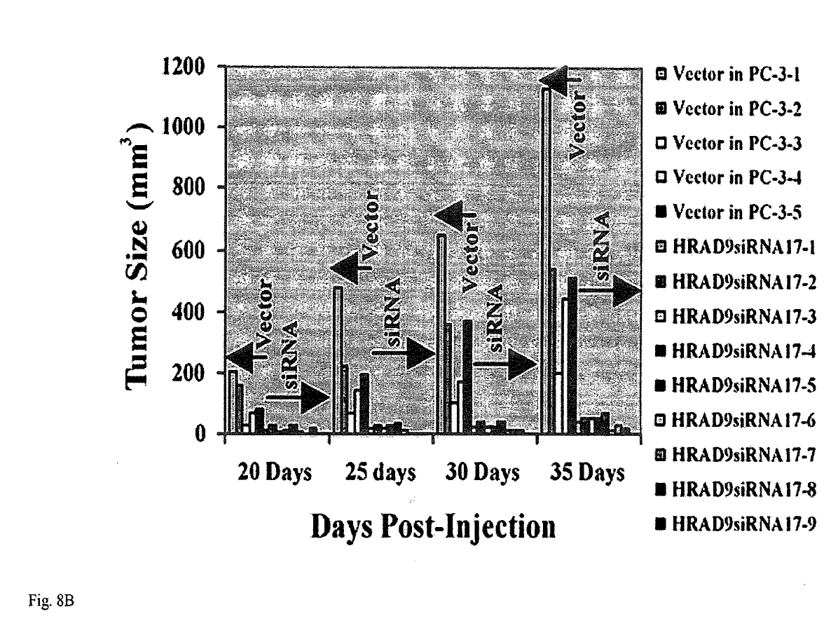

The siRNA was most effective in reducing levels of HRAD9 protein in DU145 cells, followed by reduction in PC-3 (FIG. 7). siRNA was least effective in CWR22. Densitometric scanning of HRAD9 and beta-actin (control) bands indicates that reduction in HRAD9 levels, relative to untransfected or insertless vector controls, was by 36% for DU145 (two independent clones), 76% for PC-3 (one clone) and only by 34% for CWR22 (two different clones).

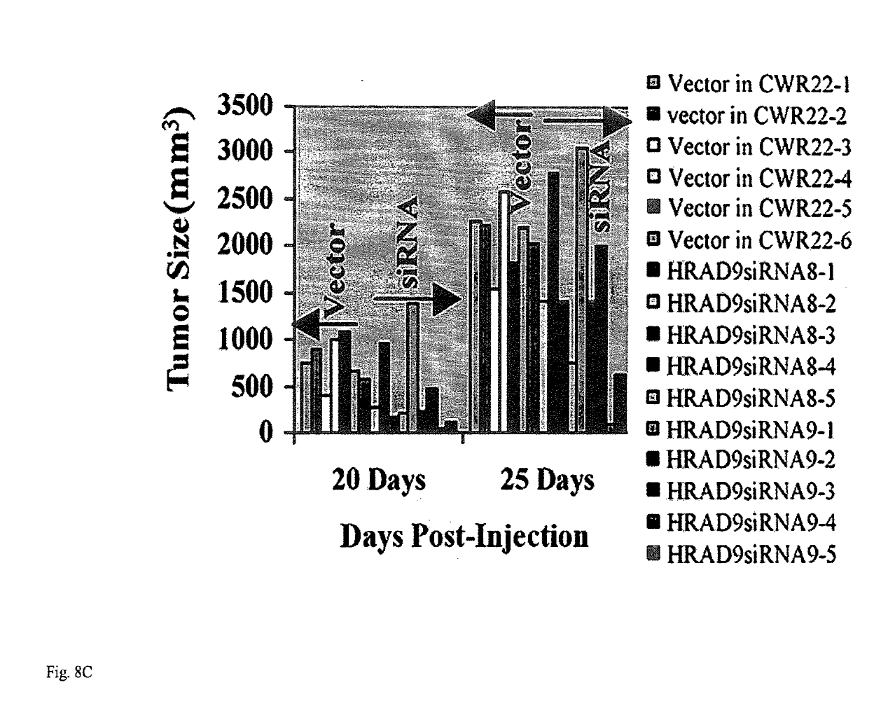

Vector or siRNA bearing cells were injected into nude mice and animals were examined for tumor development. Tumor size (mm3) versus days post-injection is presented in FIG. 8B, C, D. Mice injected with DU145 cells containing insertless vector (FIG. 8B, first 4 bars in each group) formed detectable tumors starting at day 20, which continued to grow until day 35 (last day of monitoring). However, sites injected with siRNA-containing D0145 cells contained no abnormal growths. These sites were monitored for 5 months, and still no tumors formed. Sites injected with PC-3 cells bearing insertless vector developed tumors that grew progressively during the 35 days post-injection (FIG. 8C, first five bars in each group). Interestingly, sites containing the same parental cells but with pSUPER.retro.puro Rad9 siRNA, which reduced levels of the protein significantly but not as dramatically as for D0145, developed small masses by day 20 but they stopped growing shortly thereafter and remained approximately the same size through day 35 (FIG. 8C, last seven bars of each group). In contrast, for CWR22 cells with insertless vector and high Rad9 levels, tumors grew so aggressively that by day 25 the experiment was terminated FIG. 8D, first seven bars in each group). siRNA was not very effective in reducing Rad9 protein levels in CWR22, and for most independent siRNA transfectants tumors grew aggressively at injection sites (FIG. 8D, last six bars).

Some variability in tumor size and growth rate was observed for all injections of similar cells, and this could reflect differences in mice, differences in exact numbers of cells injected, internal growth of tumors not easily measurable in vivo, or changes in Rad9 levels post-injection. However, examination of thin sections of several tumors that formed indicated that levels of Rad9 protein were similar to those observed in the parental cells in vitro. Nevertheless, it is clear that in general the more Rad9 protein present in prostate cancer cells the more avidly they form tumors when injected into nude mice, indicating a functional relationship between Rad9 abundance and prostate cancer. Furthermore, overexpression of Rad9 in the nontumorigenic, immortalized human prostate cell line PWR-1E conferred upon those cells the ability to form aberrant growths two weeks post-injection, although about two to three weeks later many began to regress. Specifically, 10 of the 14 sites injected with PWR-1E cells overproducing human Rad9 formed an abnormal growth. In contrast, no abnormal growths formed in 6 sites injected with parental PWR-1E cells, and only 1 site of 12 injected with PWR-1E containing an insertless vector control formed a small growth that regressed after 25 days. These data indicate again a functional role for Rad9 in prostate carcinogenesis.

No correlation between Rad9, AR and PSA protein levels in relation to tumorigenicity of human prostate cancer cells. Since Rad9 was found previously to interact with AR and modulate its ability to transactivate downstream target genes (25, 26), the relevance of Rad9-AR binding to tumorigenesis was examined. In particular, AR regulates the gene encoding PSA, so we examined levels of this protein in the four human prostate cancer cell lines, CWR22, DU145, LNCaP and PC-3, as a measure of AR transactivity. These four cell populations are capable of forming tumors in nude mice, have very high levels of Rad9 protein, and reduction of Rad9 abundance in DU145 as well as PC-3 reduces or eliminates the ability to form tumors in nude mice. Only CWR22 and LNCaP have AR it protein. Western analyses indicated that only LNCaP had high levels of PSA data not shown), thus demonstrating that the Rad9-AR interaction is not universally essential for prostate cancer cell tumorigenicity since the presence of AR and high levels of PSA did not correlate with ability to form tumors in nude mice. Thus, the role of Rad9 in prostate cancer is likely unrelated to its relationships with AR and ISA.

In summary, it was found that a high level of Rad9 protein is associated with prostate cancer and, furthermore, reduction in that level neutralizes the tumorigenic effects of prostate cancer cells. These results indicate that Rad9 could serve as a biomarker for prostate cancer, and, in addition, as a novel molecular target to treat the disease.

Establishing Stable Clones of HRAD9 Small Interfering RNA (siRNA) in Prostate Cancer Cells
Making HRAD9 siRNA Construct and Inducing Recombinant Virus

The chosen HRAD9 siRNA target sequence (AGGCCCGCCADCUUCACCA) (SEQ ID NO: 5) was obtained from Oligoengine Inc. and the target oligo was also synthesized by Oligoengine Inc. The pSUPER.retro.puro™ siRNA expression vector (Oligoengine Inc., WA) was used to construct a HRAD9/siRNA. The pSUPER.ret.puro™ HRAD9 siRNA plasmid was transfected to retrovirus package phi-NX cell line using lipofectamine (Invitrogen Inc.). After 24 hours, the cells were selected by puromycine 2 μg/ml in complete growth medium and incubated at 37° C. for 5 days. Then cells were split into dishes for another 2 days of incubation with puromycine. After that, the medium was changed to 5% FBS with growth medium in 30° C. for 24 hours. The culture medium was collected after centrifuging for 5 mins. at 2000 rpm and filtering through a 0.45 μm filter and saved at −80° C. as recombinant virus for later infection.

Using Recombinant HRAD9 siRNA and vector alone virus to infect prostate cancer cells—PC1, DU145, CWR22 and LNCaP prostate cancer cells were seeded at 500,000 cells per 100 mm plate in 10 mls of complete medium. 24 hours later, the growth medium was removed from the cells. 2 mls of the viral stock was added to cells in the presence of bug polybrene per ml (Chemicon International Serological Company) and incubated for 6 hours at 37° C. Then the cells were added to 8 mls of complete medium to continue culture. Three days post infection, the cells were split (1 to 5-20 diluted) into puromycine 1 μg/ml selection medium, and medium was changed every 3 to 4 days until the clones were picked up. The clone cells were cultured for amplification. Western-blotting was used to select positive clones, that is those demonstrating reduced levels of Rad9 protein.
Induced Tumor in Nude Mice HRAD9 siRNA positive clone cells at 6,000,000 cells per mouse A. Be, G. LUmmen, K. Rembrink, T. Otto, K. Metz and H. Rübben, Influence of pertussis toxin on local progression and metastasis after orthotopic implantation of the human prostate cancer cell line PC3 in nude mice, Prostate Cancer and Prostatic Diseases 1999, 2(1):36-40) in 0.2 ml of PBS were injected subcutis into the back of nude mice. When tumors did form, they were first detected after about 2 to 3 weeks post-injection. Tumor size was measured every 5 days. When tumor size reached sufficient size with necroses, the mice were sacrificed and samples were saved for further analyses. Except for LNCaP, tumors were formed after about 2 to 3 weeks. Tumor size was measured every 5 days. When tumor size reached sufficient size with necroses, the mice were killed and samples were saved for identification.

### Discussion

It is demonstrated hereinabove that four prostate cancer cell lines and 153 of 339 prostate adenocarcinomas have aberrantly high levels of Rad9 protein. There was a significant correlation between Stage of prostate adenocarcinoma and level of Rad9, where the protein was most often abundant in advanced Stages. Noncancerous prostate control PrEC cells and only 2 of 52 normal prostate tissue samples had very low levels of the protein. This is the first demonstration of a link between Rad9 abundance and prostate cancer.

Overexpression of Rad9 in prostate cancer cells or tissues suggests that it may act as an oncogene, classically for example like E2F-1 when it is expressed at high levels (35). Genetic loci associated with predisposition to prostate cancer, as well as several tumor suppressor genes and oncogenes mediating sporadic prostate cancer have been identified (36). Using an accepted mouse model, it is demonstrated hereinabove that aberrantly high abundance of Rad9 protein can be critical for tumorigenicity of prostate cancer cells since reduction in the level of the protein decreases tumor formation.

Two mechanisms responsible for high levels of Rad9 in prostate cancer cells have been identified. Evidence is provided hereinabove that, in DU145, hypermethylation of cytosines in CpG islands within the 3′ region of Rad9 intron 2 is important. Cheng and coworkers (27) describe a repressor of transcription, located within 200 bps of this intron between by 406 and 605, which can be inactivated by methylation, and the Rad9 gene of DU145 is hypermethylated in this region. Treatment with 5′-aza-2′-deoxycytidine reduces the extent of that methylation, and concomitantly reduces Rad9 levels. Therefore, these findings suggest that DU145 has abnormally high levels of Rad9 because of aberrant methylation of CpG islands within intron 2 of the gene. DNA sequence analyses of the promoter and first two exons/introns of Rad9 in DU145 (also in CWR22, LNCaP and PC-3) did not reveal a mutation. These results suggest that DU145 cells demonstrate high levels of Rad9 due to abnormal activity of an upstream regulator of Rad9, perhaps a methylase, a demethylase, or a protein that controls these enzymes. Consistent with these findings, preliminary studies indicate that high levels of Rad9 protein are also frequently associated with hypermethylation of the potential transcription suppressor in primary human prostate tumors (data not shown).

The Rad9 gene was modestly amplified in prostate cancer PC-3 cells, which could account at least in part for high levels of the encoded protein detected. Other investigators reported that Rad9 copy number was increased in certain breast tumors and contributed to increased expression (27). Gene amplification in addition to aberrant methylation is a mechanism that can regulate Rad9 protein abundance.

The relevance of the physical interaction of Rad9 and androgen receptor, with respect to influence of the former on transactivity of the latter, in the context of prostate cancer is not clear or at least not a universal, biologically significant feature of the disease. For example, LNCaP and CWR22 prostate cancer cells have androgen receptor and high levels of Rad9 protein. However, only the former cells have high levels of PSA (data not shown), which is regulated by androgen receptor. Furthermore, there is no relationship of the role of Rad9 in prostate cancer to dependence on androgen for growth or tumorigenesis since CWR22 and LNCaP are androgen dependent whereas DU145 and PC-3 are androgen independent, yet they all contain high levels of Rad9 protein and can form tumors in nude mice.

Herein it is demonstrated that high levels of Rad9 are associated with prostate cancer, and can be critical for tumorigenicity. Other investigators found that Rad9 overexpression is associated with breast cancer and nonsmall cell lung carcinoma (27, 37). Despite these results, Rad9 is likely not a universal oncogene linked to cancer of all tissues since preliminary studies indicate no correlation between abundance of Rad9 and cancer of the stomach or colon (unpublished data).

### Materials and Methods

Cells, culture conditions and 5′-aza-2′-deoxycytidine treatment. Prostate cancer cell lines, CWR22, DU145, LNCaP and PC-3, were grown at 370 C, 5% CO2 in RPM 1640 medium (Invitrogen Corp., Carlsbad, Calif.), supplemented with 10% FBS. Normal prostate epithelial cells, PrEC (Cambrex Inc., Rockland, Me.), were grown in PrEGM Bulletkit medium, serum-free (Cambrex, Inc.) at 370 C, 5% CO2.

Cells were treated with the demethylating agent 5′-aza-2′-deoxycytidine (Sigma-Aldrich Corp., St. Louis, Mo.; 0.25 mM; 200 mM stock in DMSO) as published (27). Control cells were treated with an equivalent amount of DMSO alone. Media were changed daily during the four-day treatment period. Then, cells were collected using trypsin-EDTA for DNA and protein isolation.

Prostate tissue samples. Human prostate tissue arrays were obtained from Imgenex Corp. (San Diego, Calif.) and US Biomax, Inc. (Rockville, Md.). Sample slides contained 391 human prostate tissue thin sections. The majority of sections, 335, were prostate adenocarcinomas representing Stages I through IV from different patients, and there were four independent metastases. There were 52 normal prostate tissues, derived from a subset of the population with prostate adenocarcinomas, as well as from unrelated individuals. Dr. Harshwardhan M. Thaker, a Pathologist at Columbia University, checked to confirm that the specimens contained cancerous or normal prostate tissues as indicated by the commercial vendors. No patient identifiers were available.

Southern blotting, western blotting, quantitative RT-PCR and immunohistochemistry. For Southern blotting, genomic DNA was isolated from prostate cells. DNA (5 μg) was digested with EcoRI, fractionated on a 0.7% agarose gel, transferred to a nylon membrane and hybridized to a 32P-labeled full-length HRAD9 cDNA or beta-actin (internal control) 540 bp PCR product (primers for amplification were 5′-GTTGCTATCCAGGCTGTGC-3′ (SEQ ID NO:6) and 5′-GCATCCTGTCGGCAATGC-3′ (SEQ ID NO:7); see reference 2%) probe at 650 C overnight. The membrane was then washed using a standard protocol (Amersham Biosciences, Piscataway, N.J.). X-ray film was exposed to the membrane for one to two days. After developing the film, band intensities were analyzed with Image J (NIH, Bethesda, Md.).

For Western blotting, 2×105 pelleted prostate cells were lysed in 1×SDS sample buffer with 5% 2-Mercaptoethanol. Twenty microliter volumes were taken for each sample, boiled 5 min and subjected to SDS-polyacrylamide gel electrophoresis (4-20%, Invitrogen Corp.). After electrophoresis, samples were transferred to a PVDF membrane by electroblotting for 30 min. Blots were blocked by 5% non-fat milk and probed with monoclonal mouse anti-human Rad9 antibody (BD Biosciences, Franklin Lakes, N.J.) and goat anti-human actin antibody (Santa Cruz Biotechnology, Inc., Santa Cruz, Calif.), followed by addition of secondary antibody conjugated with HPR. ECL western blotting substrate (Pierce Inc., Rockford, Ill.) was used to detect protein bands.

For quantitative real time PCP, RNA was isolated from prostate cells using TRIzol (Invitrogen Corp.), as per the manufacturer. RNA (5 μg) was reverse transcribed into cDNA using the superscript II First Strand Synthesis system (Invitrogen Corp.). cDNA was used to amplify HRAD9 by PCR. DNA primers for the reaction were 5′-TCTGCCTATGCCTGCTTTCTCT-3′ (SEQ ID NO:8) and 5′-AGCGGAAGACAGACAGGAAAGAC-3′ (SEQ ID NO:9). CAPON served as an internal reference gene to normalize measurement of HRAD9 RNA abundance. DNA primers for GAPDH were purchased from Super Array, Inc. (UNiGene#: Hs.544577, RefSeq Accession; NM_{—}002046.2). Quantitative real time PCR was performed in 25 μl, using the SYbR Green PCR Master Mix kit (Applied Biosystems, Foster City, Calif.). PCR trials were carried out in triplicate in the Applied Biosystems 7300 Real Time PCR system (ABI). PCR conditions were 1 cycle of 500 C for 2 min, 1 cycle of 950 C for 10 min and 55 cycles of 950 C for 15 s, 600 C for 30 s, and 720 C for 30 s. Relative quantification of HRAD9 RNA abundance was analyzed by the comparative threshold cycle (Ct) method (29).

Prostate cells were split into 2-well chamber slides in preparation for immunohistochemical staining. When cells reached 50% confluence, they were fixed with 4% PFA and 0.02% NP-40 overnight, then washed in PBS (1×) with 0.01% Tween 20. VECTASTAIN elite ABC kit was used for immunostaining (Vector Laboratories, Inc., Burlingame, CA). Cells were washed twice for 5 min after fixation. For quenching of endogenous peroxides, slides were immersed in 3% hydrogen peroxide solution for 5 min and washed again twice for 5 min, then blocked with normal serum (1:50 from ABC kit; Vector Laboratories, Inc.) at room temperature for 30 min. Slides were incubated with monoclonal anti-human Rad9 primary antibody (1 to 100 dilution; BD Biosciences) overnight at 40 C and washed 3×5 min, incubated with biotin-conjugated secondary antibody for 30 min at room temperature and washed again for 3×5 min, then further incubated with Avidin-Biotin complexes for 30 min at room temperature. After washing 3×5 min, slides were incubated in fresh diaminobenzidine tetrahydrochloride (DAB) substrate solution for 5 min. The reaction was stopped by washing in tap water. Counterstaining was performed with Meyer's hematoxylin. Dehydration was carried out in 75%, 80%, 95% and 100% ethanol, sequentially, followed by soaking in xylene, and mounting cover slips with Permount.

For immunostaining of prostate tissue array slides, deparaffinization, hydration and immunohistochemistry protocols supplied by Imgenex were followed, then slides were deparaffinized in Safeclear II reagent (Fisher Scientific Co., Middletown, Va.) and rehydrated with 100% ethanol, then 95%, 75%, and finally 50% ethanol. Citrate buffer (0.01 M, PH 6.0) was used for antigen retrieval. Immunohistochemical staining procedures, as described above, were then followed.

Methylation at CpG islands. Genomic DNA from prostate cells was extracted using the DNeasy Tissue Kit (Qiagen, Inc., Valencia, Calif.). Primary normal and cancerous biopsy samples were scraped from slides after deparaffinization and processed for DNA purification, as per cell samples.

Methylation status of HRAD9 CpG islands was determined using the sodium bisulfite sequencing method. Genomic DNA (2 μg) was subjected to bisulfate modification using the EZ DNA Methylation Kit (Zymo Research Corp., Orange, Calif.), following the manufacturer's instructions. Bisulfate-treated DNA (4 μl) was amplified in 25 μl containing 1× reaction buffer, 3 mM MgCl₂, 0.2 mM each dNTP, 1.5 units Expand high fidelity Taq DNA polymerase (Roche, Indianapolis, Ind.) and 0.3 μM each forward and reverse primer. HRAD9 CpG islands span over 900 bp from the promoter region to intron 2 of the gene (27, 30). This region was amplified as two overlapping DNA fragments. The first region contained the proximal HRAD9 promoter and part of axon 1 from −421 to 13 (“A” in the start codon ATG is +1). The second region spanned from −8 to 559, and included part of the proximal promoter through most of intron 2. These two regions were amplified by nested PCR. First round PCR condition was hot start at 95° C. for 5 min, then 40 cycles at 94° C. for 30 s, 520 C for 45 s, and 720 C for 1 min, and a 10 min final extension at 72° C. One microliter was then diluted 400 fold and used as template in the nested PCR reaction. The PCR condition was hot start at 95° C. for 5 min, 35 cycles at 94° C. for 30 s, 56° C. for 30 s, 72° C. for 1 min, and a 7 min final extension at 72° C. Nested PCR products were purified with the GFX PCR DNA and Gel Band purification Kit (Amersham Biosciences), then cloned into pGEM-T Easy (Promega, Madison, Wis.). T-A plasmid constructs were transformed into E. coli JM109 competent cells (Promega). Ten independent bacterial colonies, derived from each construct, were picked, purified, and DNA was isolated from them using the Miniprep Kit (Qiagen). CNA samples were sent to the Columbia University DNA Sequence Facility (New York, N.Y.) for sequence determination, using a SP6 primer (pGEM-T Easy vector has T7 and SP6 promoters). Cytosine methylation of each CpG island dinucleotide was determined by checking the cytosine signal at CpG island positions. Primer pairs for PCR and nested PCR were

(Rad9-PF; −420 to −398)
(SEQ ID NO: 10)
5′-TAAGTGGGTGATTTTAGAGAGTT-3′
and
(Rad9-PR; +159 to +182)
(SEQ ID NO: 11)
5′-CCTCCAAAAATTCCAAATAAAACT-3′;
(Rad9-PF; −420 to −398)
(SEQ ID NO: 12)
5′-TAAGTGGGTGATTTTAGAGAGTT-3′
and
(Rad9-PRn; −10 to +13)
(SEQ ID NO: 13)
5′-CCAAACACTTCATACTACCCCAA-3′;
and
(Rad9-IF; -43 to −24)
(SEQ ID NO: 14)
5′-GGAGAGTTGGGTAGTGTTGG-3′;
(Rad9-IR; +619 to +639)
(SEQ ID NO: 15)
5′-CCTTCATCAAAATCTTACAAC-3′;
(Rad9-IFn; -8 to +16)
(SEQ ID NO: 16)
5′-GGGGTAGTATGAAGTGTTTGGTTA-3′
and
(Rad9-IRn; +535 to +559)
(SEQ ID NO: 17)
5′-CCCAACCCTCTAACTACTTCTACTC-3′.

Tumorigenicity of human prostate cells in nude mice. Human prostate cells (6×106 per test site) were suspended in sterile PBS (0.2 ml) and injected into the back of nude mice subcutis. Nude mice were Harlan Sprague Dawley, Inc. Indianapolis, Ind.), Nu/Nu males, four weeks old. Each mouse was injected at two to four different sites, to reduce the number of animals needed. In pilot studies with normal (PrEC) and tumor (CWR22, DU145, PC-3) cells there was no detectable difference in tumor formation whether one or up to four sites per animal were injected. Tumors were detectable, initially assessed and measured usually after two to three weeks post-injection. Tumor size was measured every five days with a vernier caliper, by two investigators blinded such that mouse identities were coded and unknown until the experiment ended. Tumor volume was calculated based on the average of the two sets of measurements. Tumors were dissected from animals and saved in 10% formaldehyde. Tumor thin sections were stained with haematoxylin and eosin (H&E) to view histological morphology, and with antibodies against human epithelial cell specific markers (i.e., cytokeratin 5, 18, 19; Sigma), using the ABC kit (Vector Laboratories) and procedures employed to stain tissue microarrays, to confirm their human origin.

HRAD9 siRNA viral vector, recombinant virus, and infection of prostate cancer cells. The HEADS siRNA target sequence (AGGCCCGCCAUCUUCACCA) (SEQ ID NO:5) was designed and synthesized by Oligoengine, Inc. pSUPER.retro.puro siRNA expression vector (Oligoengine, Inc.) was used to construct a HRAD9 siRNA plasmid, which was transfected into the retrovirus packaging phi-NX cell line employing lipofectamine (Invitrogen Corp.). After 24 hours, cells were challenged with puromycin (2 μg/ml) in complete growth medium and incubated at 37° C. for 5 days. Cell cultures were split into dishes for another 2 days of incubation with the drug. Afterwards, medium was replaced with fresh growth medium containing 5% FBS, and cells were incubated at 30° C. for 24 hours. Culture medium was collected after centrifuging (5 min, 2000 rpm) and passing through 0.45 μm filters, saved at −80° C. as a recombinant virus stock, then used for infection.

Prostate cancer cells were seeded at 500,000 per 100 mm plate in 10 ml of complete medium. Twenty-four hours later, growth medium was removed. Two ml of viral stock was added to cells in the presence or 10 μg polybrene per ml (Chemicon International) and incubated for 6 hours at 37° C. Then, complete medium (8 ml) was added. Three days post-infection, cells were split (1 to 5-20 dilution) into selection medium containing puromycin (1 μg/ml), and medium was changed every 3 to 4 days until surviving clones were picked, then expanded. Western blotting with Rad9 antibodies was used to assess protein levels for selection of clones demonstrating reduction in Rad9 protein abundance.

### REFERENCES

1. Suzuki H, Freije D, Nusskern D R, et al. Interfocal heterogeneity of PTEN/MMAC1 gene alterations in multiple metastatic prostate cancer tissues. Cancer Fee 1998; 58:204-9.
2. Ellwood-Yen K, Graeber T G, Wongvipat J, et al. Myc-driven murine prostate cancer shares molecular features with human prostate tumors. Cancer Cells 2003; 4:223-38.
3. DiGiovanni J, Kiguchi K, Frijhoff A, et al. Deregulated expression of insulin-like growth factor 1 in prostate epithelium leads to neoplasia in transgenic mice. Proc Natl Acad Sci USA 2000; 97:3455-60.
4. Terry S, Yang X, Chen M W, Vacherot F, Buttyan R. Multifaceted interaction between the androgen and Wnt signaling pathways and the implication for prostate cancer. J Cell Biochem 2006; in press).
5. Kasper S. Survey of genetically engineered mouse models for prostate cancer: analyzing the molecular basis of prostate cancer development, progression, and metastasis. J Cell Biochem 2005; 94:279-97.
6. Klein R D. The use of genetically engineered mouse models of prostate cancer for nutrition and cancer chemcprevention research. Mutat Res 2005; 576:111-9,
7. Paris P L, Andaya A, Fridly J, et al. Whole genome scanning identifies genotypes associated with recurrence and metastasis in prostate tumors. Human Mol Genet. 2004; 13:130
9. Lieberman H C, Hopkins K M, Nass M, Demetrick D, Davey S. A human homolog of the Schizosaccharomyces pombe rad9+ checkpoint control gene. Proc Natl Acad Sci USA 1996; 93:13890-95.
9. Lieberman H B. Rad9, an evolutionarily conserved gene with multiple functions for preserving genomic integrity. J Cell Biochem 2006; 97:690-697.
10. Komatsu K, Miyashita T, Hang H, et al. Human homologue of S. pombe Rad9 interacts with Bcl-2/Bci-XL and promotes apoptosis. Nature Cell Biol 2000; 2:1-6.
11. Hopkins K M, Auerbach W, Wang X Y, et al. Deletion of mouse Rad9 causes abnormal cellular responses to DNA damage, genomic instability, and embryonic lethality. Mol Cell Biol 2004; 16:7235-48.
12. Bessho T, Sancar A. Human DNA damage checkpoint protein hRAD9 is a 3′ to 5′ exonuclease. J Biol Chem 2000; 275:7451-4.
13, Yin Y, Zhu A, Jin Y J, et al. Human RAD9 checkpoint control/proapoptotic protein can activate transcription of p21. Proc Natl Acad Sci USA 2004; 101:8864-69.
14. Lieberman H B, Yin Y. A novel function for human Rad9 protein as a transcriptional activator of gene expression. Cell Cycle 2004; 3:1006-10.
15. Ishikawa K, Ishii H, Murakumo Y, Mimori K, Kobayashi M, Yamamoto K I, Mori M, Nishino H, Furukawa Y, Ichimura K. Rad9 modulates the P21WAF1 pathway by direct association with p53, BMC Mol Biol 2007; 18:37-46,
16. Lindsey-Boltz L A, Wauson E M, Craves L A, Sancar A. The human Rad9 checkpoint protein stimulates the carbamoyl phosphate synthetase activity of the multifunctional protein CAD. Nucl Acids Res 2004; 32:4524-30.

17. Toueille M, El-Andaloussi N, Frouin I, et al. The human Rad9/Rad1/Hus1 damage sensor clamp interacts with DNA polymerase beta and increases its DNA substrate utilisation efficiency: implications for DNA repair. Nucl. Acids Res 2004; 32:3316-24.
18. Wang W, Brandt P, Rossi M L, et al. The human Rad9-Rad1-Hus1 checkpoint complex stimulates flap endonuclease 1. Proc Natl Aced Sci USA 2004; 101:16762-67,
19. Friedrich-Heineken E, Toueille M, Tännler B, et al. The two DNA clamps Rad9/Rad1/Hus1 complex and proliferating cell nuclear antigen differentially regulate Flap Endonuclease 1 activity. J Mol Biol 2005; 353:980-9.
20. Halt C E, Wang W, Keng P C, Bambara R A Evidence that DNA damage detection machinery participates in DNA repair. Cell Cycle 2005; 4:529-32.
21. Smirnove E, Toueille M, Markkanen E, Hübsche U. The human checkpoint sensor and alternative DNA clamp Rad9/Rad1/Hus1 modulates the activity of DNA ligase I, a component of the long patch base excision repair machinery. Biochem J 2005; 389:13-7.
22, Wang W, Lindsey-Boltz L A, Sancar A, Bambara R. Mechanism of stimulation of human DNA ligase I by the rad9-rad1-hus1 checkpoint complex. J Biol Chem 2006; 231:20865-72.
23. Gembke A, Toueille M, Smirnova E, Foltz R, Ferrari E, Giuseppe Villani G, Hübscher U. The checkpoint clamp, Rad9-Rad1-Hus1 complex, preferentially stimulates the activity of apurinic/apyrimidinic endonuclease 1 and DNA polymerase (beta) in long patch base excision repair. Nuc Acids Res 2007; 35:2596-608,
24. Pandita R K, Sharma G, Laszlo A, et al. Mammalian Rad9 plays a role in telcmere stability, S— and O₂-phase specific cell survival and homologous recombinational repair. Mol Cell Biol 2006; 26:1850-64.
25. Wang L, Hsu C L, Ni J, et al. Human checkpoint protein hRad9 functions as a negative coregulator to repress androgen receptor transactivation in prostate cancer cells. Mol Cell Biol 2004; 24:2202-13.
26. Hsu C-L, Chen Y-L, Ting H-J, et al. Androgen receptor (AR) NH2- and COON-terminal interactions result in the differential influences on the AR-mediated transactivation and cell growth. Mol Endocrinol 2005; 19:350-61.
27. Cheng C M, Chow L W, Loo W T, Than T K, Chan V. The cell cycle checkpoint gene Rad9 is a novel oncogene activated by 11q13 amplification and DNA methylation in breast cancer. Cancer Res 2005; 65:8646-54.
29. Zhao Y L, Piao C Q, Wu L J, et al. Differentially expressed genes in asbestos-induced tumorigenic human bronchial epithelial cells: implication for mechanism. Carcinogenesis 2000; 21: 2005-10.
29. Shao G, Berenguer J, Borczuk A C, Powell C A, Hei T K, Zhao Y. Epigenetic inactivation of Betaig-h3 gene in human cancer cells. Cancer Res 2006; 66:4566-73,
30. Takai C, Jones P A. Comprehensive analysis of CpG island in human chromosomes 21 and 22. Proc Natl Acad. Sci. USA 2002; 99:3740-5.
31. Strathdee G, Sim A, Brown R. Control of gene expression by CpG island methylation in normal cells. Biochem Soc Trans 2004; 22:913-5.
32. Klose R. J, Bird A P. Genomic DNA methylation: the mark and its mediators. Trends Biochem Sci 2006; 31:89-97.
33. Bex A, Lummen G, Rembrink K, Otto T, Metz K, Rubben H. Influence of pertussis toxine on local progression and metastasis after orthotopic implantation of the human prostate cancer cell line PC3 in nude mice. Prostate Cancer Prostatic Dis 1999; 2:36-40.
34. Zar J H. Biostatistical Analysis. 4th ed. Upper Saddle River: Prentice Hail; 1999. p. 555-7.
35. Johnson D G, Cress W D, Jakio L, Nevins J R. Oncogenic capacity of the E2F1 gene. Proc Natl Acad Sci USA 1994; 91:12923-7.
36. Shand R L, Gelmann E P. Molecular biology of prostate-cancer pathogenesis. Curt Opin Ural 2006; 16:123-31,
37. Maniwa Y, Yoshimura M, Bermudez V P, et al. Accumulation of hRad9 protein in the nuclei of nonsmall cell lung carcinoma cells. Cancer 2005; 103:126-32.

1-57. (canceled)

58. A method of determining the likelihood that a human subject is suffering from a prostate cancer comprising:
a) obtaining a sample from the subject;
b) quantitatively determining the amount of human Rad9 or the amount of human Rad9-encoding messenger RNA in the sample; and
c) comparing the amount of human Rad9 or human Rad9-encoding messenger RNA determined in step b) with a reference amount for a human subject not suffering from a prostate cancer,
wherein an amount of human Rad9 or human Rad9-encoding messenger RNA determined in step b) greater than the reference amount indicates that the subject is likely suffering from a prostate cancer.

59. The method of claim 58, wherein the amount of human Rad9 is quantitatively determined and the determination is made using an immunochemical, immunofluorescent or immunoradiological method.

60. The method of claim 58, wherein the human Rad9 comprises consecutive amino acid residues, the sequence of which is set forth in SEQ ID NO:1, SEQ ID NO:2 or SEQ ID NO:3.

61. the method of claim 58, wherein the amount of human Rad9-encoding messenger RNA is quantitatively determined and the determination is made using a reverse transcriptase polymerase chain reaction.

62. the method of claim 58, wherein the human Rad9-encoding messenger RNA comprises consecutive ribonucleotides, the sequence of which is the reverse transcript of the DNA sequence set forth in SEQ ID NO:4.

63. A method of determining if a prostate cancer of a human subject is metastatic comprising:
a) obtaining a sample from the subject;
b) quantitatively determining the amount of human Rad9 or the amount of human Rad9-encoding messenger RNA in the sample; and
c) comparing the amount of human Rad9 or human Rad9-encoding messenger RNA determined in step b) with a reference amount for a human subject whose prostate cancer is not metastatic,
wherein an amount of human Rad9 or human Rad9-encoding messenger RNA determined in step b) greater than the reference amount indicates that the subject's cancer of the prostate is metastatic.

64. The method of claim 63, wherein the amount of human Rad9 is quantitatively determined and the determination is made using an immunochemical, immunofluorescent or immunoradiological method.

65. The method of claim 63, wherein the human Rad9 comprises consecutive amino acid residues, the sequence of which is set forth in SEQ ID NO:1, SEQ ID NO:2 or SEQ ID NO:3.

66. the method of claim 63, wherein the amount of human Rad9-encoding messenger RNA is quantitatively determined and the determination is made using a reverse transcriptase polymerase chain reaction.

67. the method of claim 63, wherein the human Rad9-encoding messenger RNA comprises consecutive ribonucleotides, the sequence of which is the reverse transcript of the DNA sequence set forth in SEQ ID NO:4.


SEQUENCE LISTING (17 sequences)

SEQ ID NO: 1 - protein, 391 aa, homo sapiens
MKCLVTGGNVKVLGKAVHSLSRIGDELYLEPLEDGLSLRTVNSSRSAYACFLFAPLFFQQYQAATPGQDLQRCKILMKSFLSVFRSLAMLEKTVEKCCISLNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 2 - protein, 391 aa, Homo sapiens
MKCLVTGGNVKVLGKAVHSLSRIGDELYLEPLEDGLSLRTVNSSRSAYACFLFAPLFFQQYQAATPGQDLLRCKILMKSFLSVFRSLAMLEKTVEKCCISLNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 3 - protein, 391 aa, homo sapiens
MKCLVTGGNVKVLGKAVHSLSRIGDELYLEPLEDGLSLRTVNSSRSAYACFLFAPLFFQQYQAATPGQDLLRCKILMKSFLSVFRSLAMLEKTVEKCCIALNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 4 - DNA, 2128 bp, homo sapiens
gggccggcaggggcggtgcgcgggaagggaccccggacccggaggtcgcggagagctgggcagtgttggccgctggcggagcgctggggcagcatgaagtgcctggtcacgggcggcaacgtgaaggtgctcggcaaggccgtccactccctgtcccgcatcggggacgagctctacctggaacccttggaggacgggctctccctccggacggtgaactcctcccgctctgcctatgcctgctttctctttgccccgctcttcttccagcaataccaggcagccacccctggtcaggacctgctgcgctgtaagatcctgatgaagtctttcctgtctgtcttccgctcactggcgatgctggagaagacggtggaaaaatgctgcatctccctgaatggccggagcagccgcctggtggtccagctgcattgcaagttcggggtgcggaagactcacaacctgtccttccaggactgtgagtccctgcaggccgtcttcgacccagcctcgtgcccccacatgctccgcgccccagcacgggttctgggggaggctgttctgcccttctctcctgcactggctgaagtgacgctgggcattggccgtggccgcagggtcatcctgcgcagctaccacgaggaggaggcagacagcactgccaaagccatggtgactgagatgtgccttggagaggaggatttccagcagctgcaggcccaggaaggggtggccatcactttctgcctcaaggaattccgggggctcctgagctttgcagagtcagcaaacttgaatcttagcattcattttgatgctccaggcaggcccgccatcttcaccatcaaggactctttgctggacggccactttgtcttggccacactctcagacaccgactcgcactcccaggacctgggctccccagagcgtcaccagccagtgcctcagctccaggctcacagcacaccccacccggacgactttgccaatgacgacattgactcttacatgatcgccatggaaaccactataggcaatgagggctcgcgggtgctgccctccatttccctttcacctggcccccagccccccaagagccccggtccccactccgaggaggaagatgaggctgagcccagtacagtgcctgggactcccccacccaagaagttccgctcactgttcttcggctccatcctggcccctgtacgctccccccagggccccagccctgtgctggcggaagacagtgagggtgaaggctgaaccaagaacctgaagcctgtacccagaggccttggactagacgaagccccagccagtggcagaactgggtctctcagccctggggatcagaaaggtgggcttgctggagctgagctgtttcactgcctctcgcaggccccagctggctgtcactgtaaagctgtcccacagcggtcgggcctgggccgttatctccccacaacccccagccaatcaggactttccagacttggccctgaactactgacgttcctacctcttatttctcattgagcctcaggctatactccagctggccaaggctggaaacctgtctccctcaggctcaccttcctaaggaaaatgtcatagtaggtgctgctggcccctggtgatccagcttctctgccaatcatgacctgttccttcctgaagtcctgggcatgcatctgggacccccgtggagctgacaagttttccttgctttcctgatactctttggcgctgacttggaattctaagagccttggacccgagtgtgtggctagggttgccctggctggggcccggtgccgagactcccaagcggctctgtgcagaagagctgccaggcagtgtcttagatgtgagacggaggccatggcgagaatccagctttgacctttattcaagagaccagatgggttgccccaggatccggctgccagccctgaggccaagcacggctggagacccacgacctggcctgccgttgccctgagctgcagcctcggccccaggatcctgctcacagtcaccgcaggtgcaggcaggaagcagccctgggggactggacgctgctattgattcattaaaaaaagaaaagaaaaatacaaaaaaa

SEQ ID NO: 5 - RNA, 19 nt, artificial sequence
  siRNA directed to human rad9
aggcccgccaucuucacca

SEQ ID NO: 6 - DNA, 19 bp, artificial sequence
  primer for human rad9
gttgctatccaggctgtgc

SEQ ID NO: 7 - DNA, 18 bp, artificial sequence
  primer for human beta actin
gcatcctgtcggcaatgc

SEQ ID NO: 8 - DNA, 22 bp, artificial sequence
  primer for human rad9
tctgcctatgcctgctttctct

SEQ ID NO: 9 - DNA, 23 bp, artificial sequence
  primer for human rad9
agcggaagacagacaggaaagac

SEQ ID NO: 10 - DNA, 23 bp, Artificial Sequence
  primer for human rad9
taagtgggtgattttagagagtt

SEQ ID NO: 11 - DNA, 24 bp, Artificial Sequence
  primer for human rad9
cctccaaaaattccaaataaaact

SEQ ID NO: 12 - DNA, 23 bp, Artificial Sequence
  primer for human rad9
taagtgggtgattttagagagtt

SEQ ID NO: 13 - DNA, 23 bp, Artificial Sequence
  primer for human rad9
ccaaacacttcatactaccccaa

SEQ ID NO: 14 - DNA, 20 bp, Artificial Sequence
  primer for human rad9
ggagagttgggtagtgttgg

SEQ ID NO: 15 - DNA, 21 bp, Artificial Sequence
  primer for human rad9
ccttcatcaaaatcttacaac

SEQ ID NO: 16 - DNA, 24 bp, Artificial Sequence
  primer for human rad9
ggggtagtatgaagtgtttggtta

SEQ ID NO: 17 - DNA, 25 bp, Artificial Sequence
  primer for human rad9
cccaaccctctaactacttctactc